(19) United States
(12) Patent Application Publication
(10) Pub. No.: US 2017/0258407 A1
(43) Pub. Date: Sep. 14, 2017

(54) BIOLOGICAL INFORMATION PROCESSING SYSTEM, SERVER, BIOLOGICAL INFORMATION ESTIMATION METHOD, AND WEARABLE DEVICE

(71) Applicant: SEIKO EPSON CORPORATION, Tokyo (JP)

(72) Inventors: Tsubasa SHIRAI, Shiojiri-shi (JP); Hiroyuki ISOGAI, Chino-shi (JP)

(73) Assignee: SEIKO EPSON CORPORATION, Tokyo (JP)

(21) Appl. No.: 15/453,076

(22) Filed: Mar. 8, 2017

(30) Foreign Application Priority Data
Mar. 14, 2016 (JP) 2016-049633

(51) Int. Cl. A61B 5/00; A61B 5/026; A61B 5/01; A61B 5/021; A61B 5/11; A61B 5/024

(52) U.S. Cl. CPC A61B 5/7275; A61B 5/1118; A61B 5/681; A61B 5/02438; A61B 5/01; A61B 5/021; A61B 5/026; A61B 5/4806

(57) ABSTRACT

A biological information processing device includes: an information acquisition unit which acquires body motion information of a subject from a body motion sensor; and a processing unit which specifies an activity intensity of the subject and an activity time corresponding to the activity intensity, based on the body motion information, and estimates biological information of the subject, based on the activity time corresponding to the activity intensity.

### BACKGROUND

1. Technical Field

The present invention relates to a biological information processing system, a server, a biological information estimation method, and a wearable device.

2. Related Art

Athletes competing in running, cycling and the like have come to carry out heart rate training, for example, for the management of exercise load using biological information such as pulse rate. The objective of introducing the management of exercise load using biological information is to accurately grasp the load applied to the body, from within the body using body information, and thus to achieve improvement in efficiency of training and prevention of injuries.

Against this background, measures of acquiring pulse rates have been advancing. Up to now, measurement using a so-called heart rate sensor worn around the chest with a belt has been the mainstream method. However, a sensor that is worn around the arm and thus can acquire pulse rates has been made commercially available, improving convenience for athletes. This has enabled widespread use of heart rate training as a more familiar training method. However, the pulse wave sensor (biological sensor) worn around the arm has a problem that pulse rates cannot be measured when temperature is low or when the degree of contact between the pulse wave sensor and the arm is low. For example, if the user (subject) cannot check his/her pulse rate during exercise, the user cannot properly carry out heart rate training or the like.

Particularly the inability to measure pulse rates at the time of low temperature cannot be prevented in some cases even if the user makes adjustments to prevent it. For example, in some cases, pulse rates can be measured immediately after the start of training, but about 30 minutes after the start of the training, the temperature of the pulse wave sensor drops due to the influence of ambient temperature and pulse rates can no longer measured.

To cope with this, JP-A-2014-236775 discloses a related-art technique in which, when the user's pulse rate cannot be measured, the pulse rate is estimated and the estimated pulse rate is presented to the user.

However, in the case of continuing exercise for a long time, for example, the pulse rate may not be constant even if the exercise is carried out under the same condition. Therefore, with the related-art technique disclosed in JP-A-2014-236775, in the case where the pulse rate cannot be measured and therefore the pulse rate is estimated as the exercise continues for a long time, the estimated pulse rate may be different from the actual pulse rate. If the user refers to the value that is different from the actual pulse rate, the user cannot properly carry out heart rate training or the like.

### SUMMARY

An advantage of some aspects of the invention is to provide a biological information processing device, a server, a biological information estimation method, and a wearable device or the like which enable more accurate estimation of biological information of a subject when the biological information of the subject cannot be acquired.

An aspect of the invention relates to a biological information processing device including: an information acquisition unit which acquires body motion information of a subject from a body motion sensor; and a processing unit which specifies an activity intensity of the subject and an activity time corresponding to the activity intensity, based on the body motion information, and estimates biological information of the subject, based on the activity time corresponding to the activity intensity.

According to the aspect of the invention, the activity intensity of the subject and the activity time corresponding to the activity intensity are specified based on the body motion information, and the biological information of the subject is estimated based on the activity time corresponding to the activity intensity. Therefore, when the biological information of the subject cannot be acquired, the biological information of the subject can be estimated more accurately than when these types of information are not used.

In the aspect of the invention, the processing unit may specify each of first to N-th activity times corresponding to each of first to N-th activity intensities of the subject (N being an integer equal to or greater than 2) and estimate the biological information, based on the respective activity times.

With this configuration, when the biological information of the subject cannot be acquired, it is possible to estimate the biological information of the subject more accurately or the like, even if the subject carries out an activity with different activity intensities.

In the aspect of the invention, the processing unit may estimate the biological information, based on the activity time corresponding to the activity intensity, if the information acquisition unit cannot acquire the biological information from a biological sensor.

With this configuration, for example, it is possible to use the biological information measured by the biological sensor when the biological information can be measured, and to use the estimated biological information or the like when the biological information cannot be measured.

In the aspect of the invention, the processing unit may estimate the biological information, based on the activity time corresponding to the activity intensity and coefficient information corresponding to the activity time.

With this configuration, it is possible to estimate the biological information or the like, in consideration of variations in the biological information that can occur according to the activity time, based on the coefficient information.

In the aspect of the invention, the processing unit may specify each of first to N-th activity times corresponding to each of first to N-th activity intensities of the subject (N being an integer equal to or greater than 2), estimate each of first to N-th coefficient information corresponding to the respective activity times of the first to N-th activity times, and estimate the biological information, based on the respective activity times and the respective coefficient information.

With this configuration, for example, when the biological information of the subject cannot be acquired, it is possible to estimate the biological information or the like, in consideration of variations in the biological information that can occur according to the activity time, based on the coefficient information, even if the subject carries out an activity with different activity intensities.

In the aspect of the invention, the processing unit may estimate the coefficient information, based on the biological information, if the information acquisition unit can acquire the biological information from a biological sensor.

With this configuration, the biological information can be acquired with higher accuracy even in a time bracket during which the biological information cannot be acquired directly from the biological sensor.

In the aspect of the invention, the information acquisition unit may acquire first period biological information and a first period activity time before a non-detection period during which the biological information cannot be acquired from the biological sensor, and acquire second period biological information and a second period activity time after the non-detection period. The processing unit may estimate the coefficient information, based on the first period biological information, the second period biological information, the first period activity time, and the second period activity time.

With this configuration, it is possible to estimate the biological information or the like, in consideration of variations in the biological information that can occur in the non-detection period of the biological information.

In the aspect of the invention, the processing unit may carry out action determination processing in which an action of the subject is determined based on the body motion information, and may change the coefficient information, based on a result of the action determination processing.

With this configuration, it is possible to change the coefficient information based on the action of the subject and estimate the biological information corresponding to the action of the subject, or the like.

In the aspect of the invention, if the action of the subject is determined as a first action, the processing unit may estimate a first type activity time during which the subject carries out the first action, and first type time coefficient information corresponding to the first type activity time, and estimate first biological information, based on the first type activity time and the first type time coefficient information. If the action of the subject is determined as a second action that is different from the first action, the processing unit may estimate a second type activity time during which the subject carries out the second action, and second type time coefficient information corresponding to the second type activity time, and estimate second biological information, based on the second type activity time and the second type time coefficient information.

With this configuration, it is possible to increase the accuracy of estimation of the first biological information corresponding to the time when the subject carries out the first action, and to increase the accuracy of estimation of the second biological information corresponding to the time when the subject carries out the second action, or the like.

In the aspect of the invention, the processing unit may carry out processing of initializing the activity time if the subject is determined as being in a stopped state, in the action determination processing.

For example, when the subject is in the stopped state, the influence on the biological information of the activity before the stop may be reduced. Therefore, by the processing of initializing the activity time, it is possible to improve the accuracy of estimation of the biological information or the like, compared with the case where the activity time is not initialized.

In the aspect of the invention, if the action of the subject is determined as a first action, the processing unit may estimate a first type activity time during which the subject carries out the first action, and first type time coefficient information corresponding to the first type activity time. If the action of the subject is determined as a second action that is different from the first action, the processing unit may estimate a second type activity time during which the subject carries out the second action, and second type time coefficient information corresponding to the second type activity time. The processing unit may estimate biological information, based on the first type activity time, the first type time coefficient information, the second type activity time, and the second type time coefficient information.

With this configuration, when the biological information cannot be acquired from the biological sensor, it is possible to estimate the biological information more accurately even if the subject caries out a plurality of types of activities.

In the aspect of the invention, the information acquisition unit may acquire post-activity biological information from the biological sensor after the lapse of a predetermined period following the stop of the activity of the subject. The processing unit may specify stamina information indicating stamina consumed by the subject in carrying out the activity, based on the post-activity biological information, and estimate biological information, based on the stamina information.

With this configuration, it is possible to increase the accuracy of estimation of the biological information or the like, in consideration of the stamina information of the subject.

In the aspect of the invention, the processing unit may generate notification information of the stamina information, based on coefficient information corresponding to the activity time.

With this configuration, the subject can carry out the activity while checking his/her stamina information, or the like.

In the aspect of the invention, the information acquisition unit may acquire the body motion information from the body motion sensor and acquire the biological information from a biological sensor. The processing unit may determine whether an activity of the subject is in a stable state or not, based on the body motion information and the biological information, and estimate the biological information, based on the activity time after it is determined that the activity of the subject is in the stable state.

With this configuration, it is possible to estimate the biological information after it is determined that the activity of the subject is in the stable state, and to improve the accuracy of estimation of the biological information, or the like.

In the aspect of the invention, the processing unit may generate non-detection period notification information indicating a period during which the biological information cannot be acquired, if the information acquisition unit cannot acquire the biological information from a biological sensor.

With this configuration, it is possible to notify the subject of the non-detection period of the biological information, or the like.

Another aspect of the invention relates to a biological information estimation method including: acquiring body motion information of a subject from a body motion sensor; specifying an activity intensity of the subject and an activity time corresponding to the activity intensity, based on the body motion information; and estimating biological information of the subject, based on the activity time corresponding to the activity intensity.

Another aspect of the invention relates to a program causing a computer to function as each component of the above configuration.

### BRIEF DESCRIPTION OF THE DRAWINGS

The invention will be described with reference to the accompanying drawings, wherein like numbers reference like elements.

FIG. 1 is an explanatory view showing an example of a system configuration according to an embodiment.

FIG. 2 is an explanatory view showing activity intensity and activity time.

FIG. 3 is an explanatory view showing the phenomenon of heart rate drift.

FIG. 4 is a flowchart explaining a flow of processing according to the embodiment.

FIG. 5 is an explanatory view showing an estimated pulse rate when the phenomenon of heart rate drift occurs.

FIG. 6 is a flowchart explaining a flow of processing when the pulse rate becomes detectable again.

FIG. 7 is a flowchart explaining a flow of processing of an estimated pulse rate correction algorithm.

FIG. 8 is an explanatory view showing an estimated pulse rate after the correction algorithm is applied.

FIG. 9 is an explanatory view showing coefficient information after the correction algorithm is applied.

FIG. 10A shows the relation between the pace of mountaineering and the elevation above sea level.

FIG. 10B is an explanatory view showing the pulse rate at the time of mountaineering.

FIG. 11 is a flowchart explaining another flow of processing according to the embodiment.

FIG. 12 is an explanatory view showing an estimated pulse rate at the time of mountaineering.

FIG. 13 is an explanatory view showing an example of display of a non-detection period of biological information.

FIG. 14 is a flowchart explaining a flow of determination processing to determine whether the activity of the subject is in a stable state or not.

FIG. 15A shows an appearance of an electronic device.

FIG. 15B shows another appearance of the electronic device.

FIG. 15C shows another appearance of the electronic device.

FIG. 16A is an explanatory view showing a biological information processing system.

FIG. 16B n is another explanatory view showing the biological information processing system.

FIG. 16C is another explanatory view showing the biological information processing system.

### DESCRIPTION OF EXEMPLARY EMBODIMENTS

Hereinafter, an embodiment will be described. The embodiment below should not unduly limit the contents of the invention described in the appended claims. Not all of the configurations described in the embodiment are necessarily essential components of the invention.

### 1. Example of System Configuration

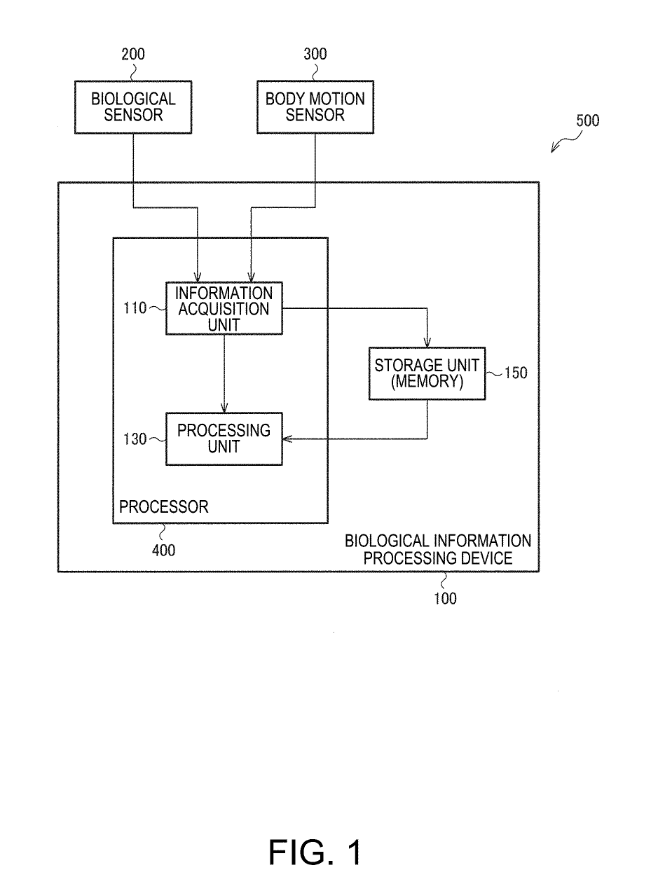

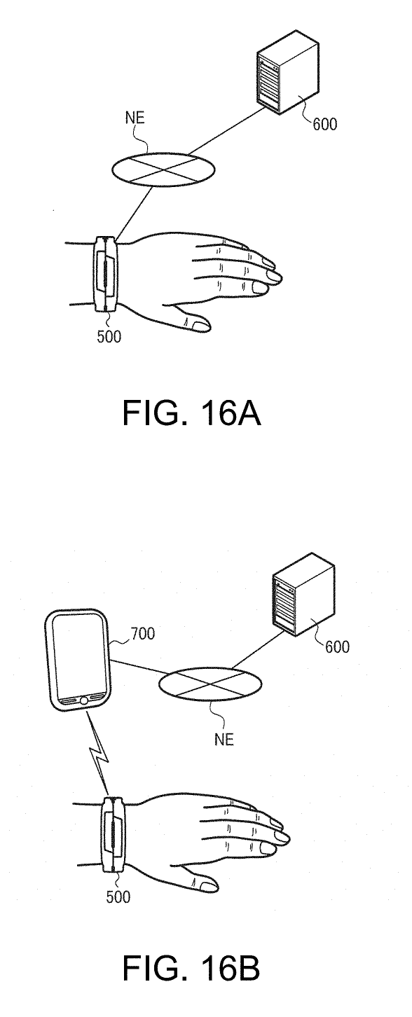

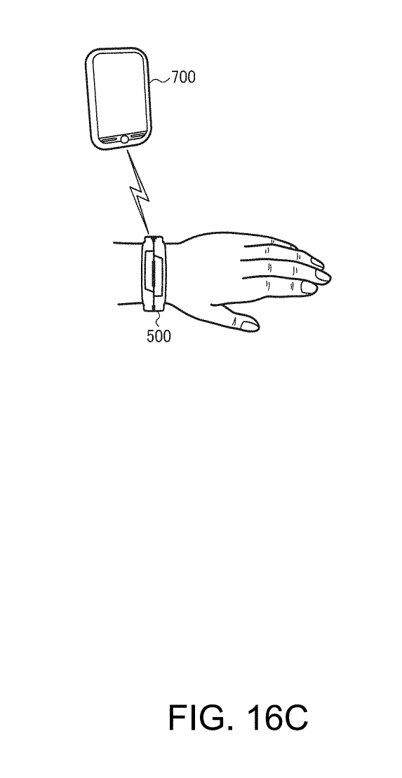

FIG. 1 shows an example of the configuration of a biological information processing device 100 (biological information processing system) according to this embodiment and an electronic device 500 including this biological information processing device 100. The biological information processing device 100 according to the embodiment includes an information acquisition unit 110, a processing unit 130, and a storage unit 150 (memory). As an example of the electronic device 500 including the biological information processing device 100, a wearable device or the like including a biological sensor 200, a body motion sensor 300 and the processing unit 130 (processor 400) may be employed. The electronic device 500 may also include a notification unit (for example, a display unit or audio output unit) or the like, not illustrated. A part or the whole of the functions of the biological information processing device 100 according to the embodiment are implemented by the wearable device, for example. A part or the whole of the functions of the biological information processing device 100 (biological information processing system) may also be implemented by an electronic device (portable electronic device) that is different from the wearable device, such as a smartphone, or by a server system. A specific example of the configuration of the biological information processing system will be described later, referring to FIGS. 16A to 16C. The biological information processing device 100 and the electronic device 500 including the biological information processing device 100 are not limited to the configuration shown in FIG. 1 and can be implemented with various modifications such as omitting apart of the components of these devices or adding other components.

Next, processing carried out by each component unit will be described. The information acquisition unit 110 acquires body motion information of a subject from the body motion sensor 300. The information acquisition unit 110 can also acquire biological information of the subject from the biological sensor 200. The body motion information is detected by the body motion sensor 300 provided in the wearable device 500, for example. Similarly, the biological information is detected by the biological sensor 200 provided in the wearable device 500 worn by the subject, for example.

In the case where the biological information processing device 100 according to the embodiment is implemented by a server system 600 and where the server system 600 acquires biological information and body motion information from the wearable device 500 worn by the user, as described later with reference to FIG. 16A, for example, the information acquisition unit 110 may be a communication unit which communicates with the wearable device 500 via a network (receiving unit which receives information from the wearable device 500). The communication unit may be a communication device such as a USB connector (communication terminal) or wireless antenna, or may be a processor or the like which controls the communication device.

The biological sensor 200 (biological sensor device) is a sensor which is provided in the wearable device 500 worn by the subject and which can acquire biological information of the subject. For example, the biological information is information indicating biological activities of the subject acquired from the biological sensor 200, such as pulse rate (heart rate), body temperature, blood pressure, amount of blood flow, activity time (hours of sleep), and activity state (exercising state). For example, if the information acquisition unit 110 acquires pulse wave information as biological information, a pulse wave sensor is used as the biological sensor 200. The pulse wave information is information about pulse waves of the subject and, for example, information indicating pulse rate (heart rate) or the like as described above. The pulse wave sensor is a sensor for detecting pulse wave information (pulse wave signal) and may be, for example, a photoelectric sensor or the like including a light emitting unit and a light receiving unit. It is known that the pulse wave sensor can be implemented by various sensors such as a photoelectric sensor or other types of sensors (for example, ultrasonic sensor). A broad range of such sensors can be applied as the pulse wave sensor in the embodiment. The biological sensor 200 may also be configured to include a blood pressure sensor, temperature sensor and the like.

The body motion sensor 300 (body motion sensor device) is a sensor which is provided in the wearable device 500 worn by the subject and which can acquire body motion information of the subject. For example, the body motion information is information indicating body motions of the subject acquired from the body motion sensor 300. The body motion information is information indicating the distance travelled by the subject, number of steps taken, stride, time of movement, moving speed, acceleration, absolute amount of change in acceleration, frequency of change in acceleration, amount of exercise, details of exercise (details of activity), difference in elevation above sea level per unit time, elevation above sea level, information acquired from a gyro sensor, absolute amount of change in angular velocity, frequency of change in angular velocity, information acquired from a geomagnetic sensor, absolute amount of change in geomagnetism, frequency of change in geomagnetism, information acquired from a barometric pressure sensor signal, or the like. In this embodiment, it is recommended that such information should be handled as multidimensional vectors. However, similar information may be omitted in order to reduce the amount of information.

As the body motion sensor 300, for example, an acceleration sensor or the like can be used. In this case, the information acquisition unit 110 acquires acceleration information (or location information) from the acceleration sensor, as body motion information. The body motion sensor 300 may also be a gyro sensor, altitude sensor, geomagnetic sensor, barometric pressure sensor or the like. The body motion sensor 300 may also be a GPS (Global Positioning System) receiver or the like, for example. In such a case, the GPS receiver (body motion sensor 300) acquires location information indicating the current location of the wearable device 500 (subject), based on radio waves transmitted from GPS satellites, and the information acquisition unit 110 acquires the location information of the wearable device 500 (subject) as body motion information.

The processing unit 130 specifies the activity intensity of the subject and the activity time corresponding to the activity intensity, based on the body motion information, and estimates biological information of the subject, based on the activity time corresponding to the activity intensity. The functions of the processing unit 130 can be implemented by hardware such as various processors (CPU or the like) and ASIC (gate array or the like), or by a program or the like. For example, in the example shown in FIG. 1, the processor 400 implements the functions of the processing unit 130 and the information acquisition unit 110. However, the embodiment is not limited to this example and various modifications can be made. For example, the biological information processing system 100 may include a plurality of processors. Of these processors, a first processor may implement the functions of the processing unit 130, and a second processor may implement the functions of the information acquisition unit 110.

In this way, in the embodiment, even if the information acquisition unit 110 cannot acquire biological information of the subject from the biological sensor 200, for example, the processing unit 130 estimates biological information of the subject, based on body motion information acquired from the body motion sensor 300. In this case, the processing unit 130 finds the activity intensity of the subject and the activity time corresponding to the activity intensity, based on the body motion information, and estimates the biological information, based on the resulting activity time. The use of the activity intensity and the activity time corresponding to the activity intensity for the estimation of biological information enables more accurate reflection of the details of the activity of the subject on the result of the estimation of biological information. Thus, when biological information of the subject cannot be acquired, the biological information of the subject can be estimated more accurately than when such information is not used. Details of the estimation of pulse rates will be described later.

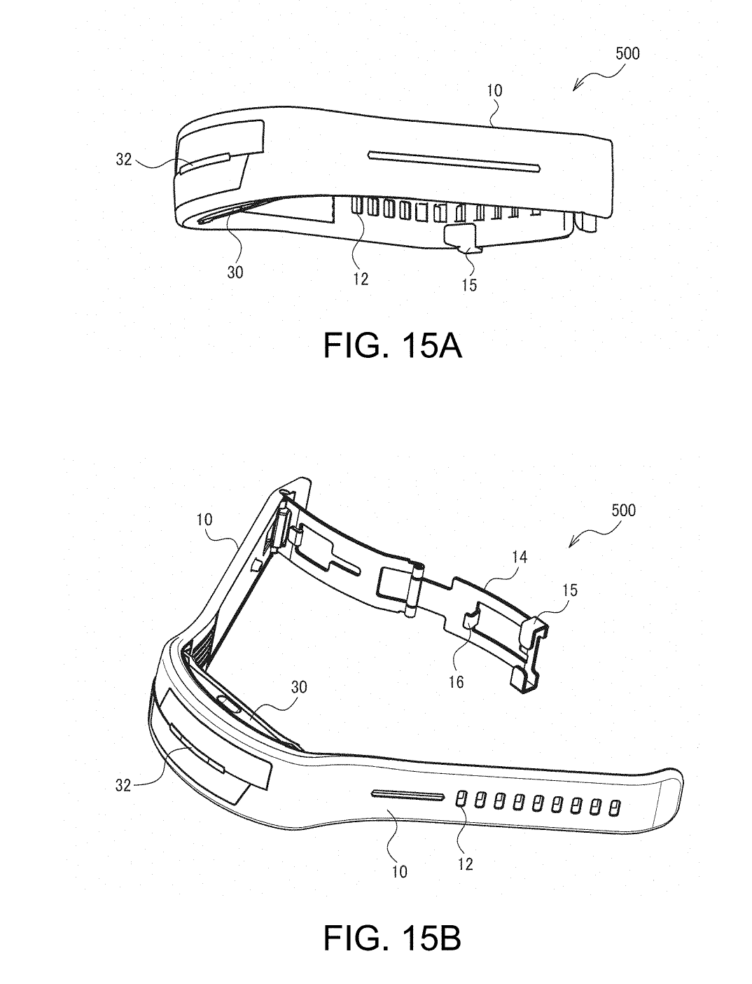

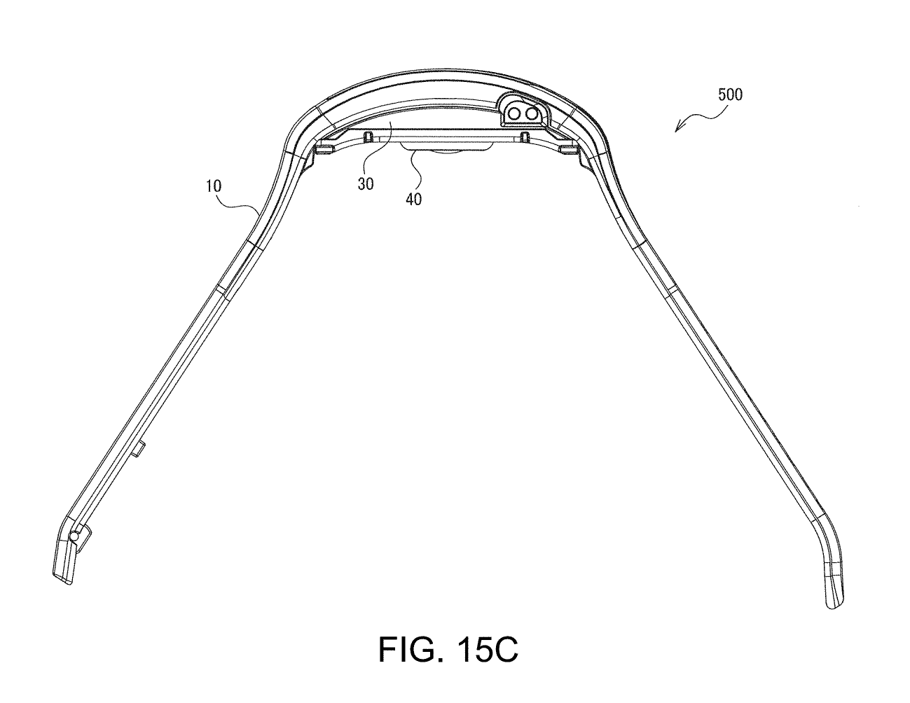

If the information acquisition unit 110 can acquire biological information from the biological sensor 200, the processing unit 130 uses the biological information measured by the biological sensor 200. If the information acquisition unit 110 cannot acquire biological information from the biological sensor 200, the processing unit 130 uses estimated biological information. For example, if biological information can be measured, the processing unit 130 can output the biological information measured by the biological sensor 200 to a presentation unit, not illustrated, so as to present the biological information to the user. If biological information cannot be measured, the processing unit 130 can output estimated biological information to a presentation unit, not illustrated, so as to present the biological information to the user. Thus, the user can constantly check biological information while wearing the electric device 500 (FIG. 15A) in this embodiment or the like.

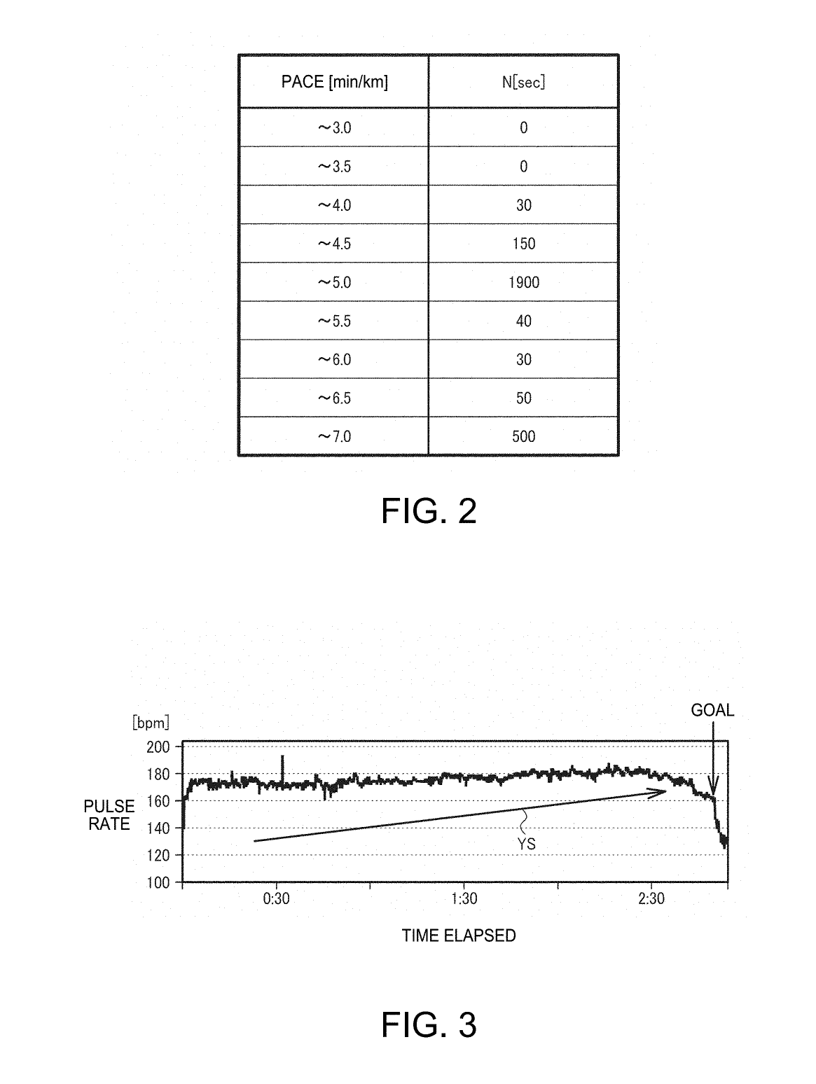

The activity intensity is a numerical value or the like indicating the intensity of the activity carried out by the subject, for example. Specifically, as shown in FIG. 2, the activity intensity is the pace (min/km) at the time of running (moving). The activity time corresponding to the activity intensity refers to the time during which an activity of that activity intensity is carried out. For example, in the example shown in FIG. 2, the pace (min/km) at the time of running is divided into predetermined ranges and the respective ranges of running pace are set as different activity intensities. For example, the running pace of up to 3.0 (min/km) is set as a first activity intensity. The running pace of 3.0 (min/km) to 3.5 (min/km) is set as a second activity intensity. The rest of the running paces is similarly divided every 0.5 (min/km). Thus, the first to ninth activity intensities are set as different levels of activity intensity. In the example shown in FIG. 2, a cumulative activity time N (sec) for which an activity of each of the nine activity intensities is carried out is measured. For example, the activity time corresponding to the fourth activity intensity, which is the running pace of 4.0 (min/km) to 4.5 (min/km), is 150 (sec). The activity time corresponding to the fifth activity intensity, which is the running pace of 4.5 (min/km) to 5.0 (min/km), is 1900 (sec).

In the embodiment, the processing unit 130 estimates the pulse rate of the subject, based on each activity time N as shown in FIG. 2. That is, the processing unit 130 specifies each of the first to N-th activity time corresponding to each of the first to N-th activity intensities of the subject (N being an integer equal to or greater than 2), and estimates biological information, based on each activity time. While N=9 holds in the example shown in FIG. 2, the embodiment is not limited to this example.

Thus, when biological information of the subject cannot be acquired, it is possible to estimate the biological information of the subject more accurately, or the like, even if the subject carries out an activity with different activity intensities.

In the embodiment, the following configuration may be employed. That is, the biological information processing device 100 includes a memory (storage unit 150) which stores information (for example, programs and various data), and a processor 400 (processing unit 130, processor made up of hardware) which operates based on the information stored in the memory. The processor 400 acquires body motion information of the subject from the body motion sensor 300 (body motion sensor device) provided in the wearable device 500. The processor 400 specifies the activity intensity of the subject and the activity time corresponding to the activity intensity, based on the body motion information, and estimates biological information of the subject, based on the activity time corresponding to the activity intensity.

In the processor (processing unit 130), for example, the functions of each component unit may be implemented by separate hardware, or the functions of the respective component units may be implemented by integrated hardware. The processor may be a CPU (Central Processing Unit), for example. However, the processor is not limited to a CPU and can be various types of processors such as GPU (Graphics Processing Unit or DSP (Digital Signal Processor). The processor may also be a hardware circuit based on ASIC (Application Specific Integrated Circuit). The memory (storage unit 150) may be a semiconductor memory such as SRAM (Static Random Access Memory) or DRAM (Dynamic Random Access Memory), or may be a register, a magnetic storage device such as hard disk device, or an optical storage device such as optical disk device. For example, the memory stores computer-readable commands. As the processor executes these commands, the functions of each component parts of the processing unit 130 are implemented. The commands in this case may be commands of a command set forming a program, or may be commands instructing the hardware circuit of the processor to execute operations.

### 2. Method in this Embodiment

Next, the method in this embodiment will be described. As described above, for example, in the case of continuing exercise for a long time, the pulse rate may not be constant even if the exercise is carried out under the same condition. An example of such a case is shown in FIG. 3. The graph shown in FIG. 3 shows the pulse rate measured when the subject runs a marathon at a substantially constant pace. The horizontal axis represents the time elapsed. The vertical axis represents the pulse rate (bpm: beats per minutes). In the example shown in FIG. 3, though the subject continues running at a substantially constant pace, the pulse rate gradually increases as indicated by an arrow YS, from the start toward the goal. For example, the pulse rate at the point of 30 minutes from the start is approximately 170 bpm, but the pulse rate after the lapse of two hours from the start is 180 bpm or above. This is because the phenomenon of heart rate drift occurs in the human body. The heart rate drift refers to a phenomenon in which the pulse rate gradually increases as the same exercise load continues to be applied to the human body. Although the mechanism of this phenomenon is not fully clarified, dehydration is pointed out as the main cause. If the pulse rate cannot be measured while such exercise is continued for a long time, when the pulse rate is estimated, the estimated pulse rate may be different from the actual pulse rate. Consequently, the user ends up carrying out heart rate training, referring to the value that is different from the actual pulse rate, and therefore cannot carry out proper heart rate training. Thus, in the embodiment, the accuracy of estimation of the pulse rate is improved by simultaneously analyzing the duration for which the exercise load is maintained in addition to the exercise load itself. The exercise load is the activity intensity described above. The duration for which the exercise load is maintained is the activity time corresponding to the activity intensity described above.

Next, a specific example of the estimation of biological information will be described. The processing unit 130 in the embodiment estimates biological information, based on the activity time corresponding to the activity intensity, and coefficient information corresponding to the activity time, which will be described later. In other words, the processing unit 130 learns the correlation between the activity time corresponding to the activity intensity and the pulse rate as biological information, and estimates the pulse rate, based on the result of learning. At this time, the information obtained as the result of learning corresponds to the coefficient information corresponding to the activity time. Hereinafter, the processing of estimating the pulse rate as biological information will be described. However, the embodiment is not limited to this and biological information other than pulse rate may be estimated.

For example, in the specific example below, the pulse rate is estimated using each activity intensity shown in FIG. 2 described above and each activity time corresponding to each activity intensity. The estimation of the pulse rate is carried out roughly by the following three steps. First, a reference pulse rate which serves as a reference is calculated from the activity intensity. Next, the amount of correction of the reference pulse rate is calculated from the activity time corresponding to the activity intensity. Then, the reference pulse rate and the amount of correction are added to find an estimated pulse rate, as shown in the following equation (1).

pulse=pulseᵢₙᵢ+f(M)+p(S) (1)

Here, the reference pulse rate is the pulse rate in the state where the phenomenon of heart rate drift had not occurred at all. In the equation (1), the value expressed by pulseᵢₙᵢ+f(M) corresponds to the reference pulse rate. Then, the influence of the phenomenon of heart rate drift is added in the form of the amount of correction p(S) to the reference pulse rate, thus finding the estimated pulse rate “pulse”. In the equation (1), pulseᵢₙᵢ is the pulse rate at rest (normal time), for example. M=(M₁ M₂ . . . )εR^{m} is information indicating the activity intensity. S=(S₁ S₂ . . . )εR^{m} is information indicating the activity time. Specifically, in the example shown in FIG. 2, M₁ is information indicating the pace of 3.0 (min/km). S₁ is information indicating the cumulative activity time for which the activity is carried out at the pace of 3.0 (min/km). M₂ to M₉ and S₂ to S₉ are similarly defined. If each of these values is found by applying linear Bayesian inference, and if f(M) is expressed by the following equation (2) and p(S) is expressed by the following equation (3), the estimated pulse rate “pulse” can be expressed in the form of linear combination as expressed by the following equation (4). In the equations (2) to (4), wₖ is coefficient information corresponding to the activity intensity or the activity time. Therefore, the correlation between the estimated pulse rate, and the activity intensity and the activity time by learning each coefficient wₖ and making updates. Here, Φ is a predetermined transform function.

$$\begin{matrix}f ( M ) = {\sum }_{k = 1}^{m} {w}_{k} {ϕ}_{k} ( {M}_{k} ) & ( 2 ) \\ p ( S ) = {\sum }_{k = 1}^{s} {w}_{k + m} {ϕ}_{k} ( {S}_{k} ) & ( 3 ) \\ pulse = {pulse}_{int} + {\sum }_{k = 1}^{m} {w}_{k} {ϕ}_{k} ( {M}_{k} ) + {\sum }_{k = 1}^{s} {w}_{k + m} {ϕ}_{k} ( {S}_{k} ) & ( 4 )\end{matrix}$$

Thus, for example, it is possible to estimate the pulse rate in consideration of the amount of increase in the pulse rate due to the phenomenon of heart rate drift, or the like. For the estimation of coefficient information and biological information, not only the above-described linear Bayesian inference but also other methods such as Kalman filter can be used.

To summary the estimation of biological information using the equations (2) to (4), the processing unit 130 specifies each of the first to N-th activity times corresponding to each of the first to N-th activity intensities of the subject (N being an integer equal to or greater than 2), as shown in FIG. 2. The processing unit 130 then estimates each of the first to N-th coefficient information corresponding to each of the first to N-th activity times and estimates biological information, based on each activity time and each coefficient information. In the example shown in FIG. 2, the coefficient information w₁ to w₉ of the activity intensity and the coefficient information w₁₀ to w₁₈ of the activity time are estimated and biological information is then estimated.

Thus, for example, when biological information of the subject cannot be acquired, it is possible to estimate the pulse rate or the like in consideration of the amount of increase in the pulse rate due to the phenomenon of heart rate drift, even if the subject carries out an activity with different activity intensities.

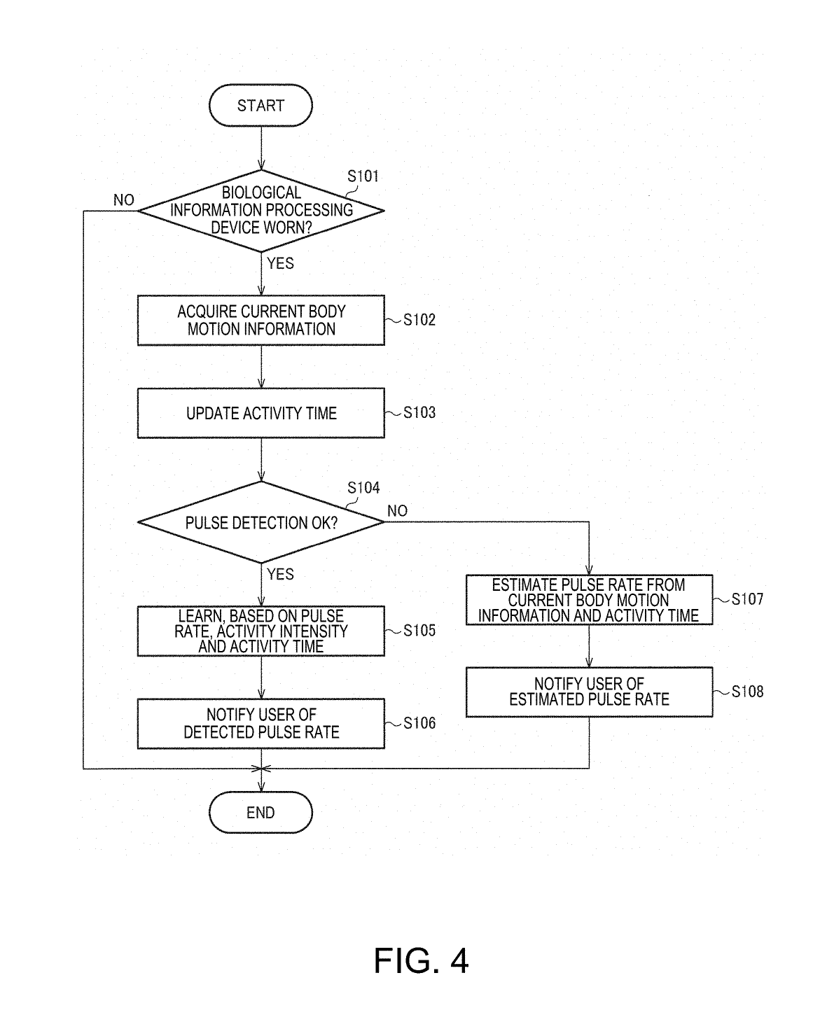

Next, a flow of processing in the embodiment will be described, referring to the flowchart of FIG. 4. First, the processing unit 130 determines whether the biological information processing device 100 (FIG. 1) or the electronic device 500 (FIG. 15A) including at least the body motion sensor 300 is worn by the subject or not (S101). If the processing unit 130 determines that the biological information processing device 100 or the electronic device 500 is not worn by the subject (S101: NO), the processing ends.

Meanwhile, if the processing unit 130 determines that the biological information processing device 100 or the electronic device 500 is worn by the subject (S101: YES), the information acquisition unit 110 acquires body motion information from the body motion sensor 300 (S102). The processing unit 130 then updates each activity time corresponding to each activity intensity of the subject, as shown in FIG. 2, based on the acquired body motion information (S103).

Next, the processing unit 130 determines whether the pulse rate is normally measured by the biological sensor 200 or not (S104). For example, in the case where the biological information processing device 100 or the electronic device 500 has a temperature sensor, not illustrated, along with the biological sensor 200 and the body motion sensor 300, the processing unit 130 determines that the pulse rate is normally measured by the biological sensor 200, only if the temperature outputted from the temperature sensor is a predetermined temperature or above.

If the processing unit 130 determines that the pulse rate is normally measured (S104: YES), the processing unit 130 carries out learning using the detected pulse rate, the activity intensity, and the activity time corresponding to the activity intensity (S105). That is, in this case, the processing unit 130 updates the coefficient information wₖ and wₖ₊ₘ in the equation (4). The processing unit 130 also notifies the user of the pulse rate measured by the biological sensor 200 (S106) and ends the processing.

Meanwhile, if the processing unit 130 determines that the pulse rate cannot be measured normally (S104: NO), the processing unit 130 estimates the pulse rate according to the equation (4) using the activity intensity and the activity time corresponding to the activity intensity (S107). In this case, the pulse rate is estimated using the coefficient information wₖ and wₖ₊ₘ updated in Step S105. The processing unit 130 then notifies the user of the estimated pulse rate (S108) and ends the processing. By thus notifying the user of the estimated pulse rate, it is possible to prevent the user's inability to check the pulse rate at all even if the biological sensor 200 cannot measure the pulse rate. In this case, it is also possible to notify the user that the provided pulse rate is not the pulse rate directly acquired from the biological sensor 200. For example, at the time of displaying the pulse rate, a “*” mark or the like may be added. Alternatively, the color of the numerical value may be changed, or the display of the numerical value may be switched on and off. The user checks such a display, determines the reliability of the displayed pulse rate, and carries out determination his/her physical condition, selection of action and the like.

### 3. Modifications

Next, a modification of the embodiment will be described. In this modification, the processing unit 130 estimates the coefficient information, based on biological information, if information acquisition unit 110 has successfully acquired the biological information from the biological sensor 200.

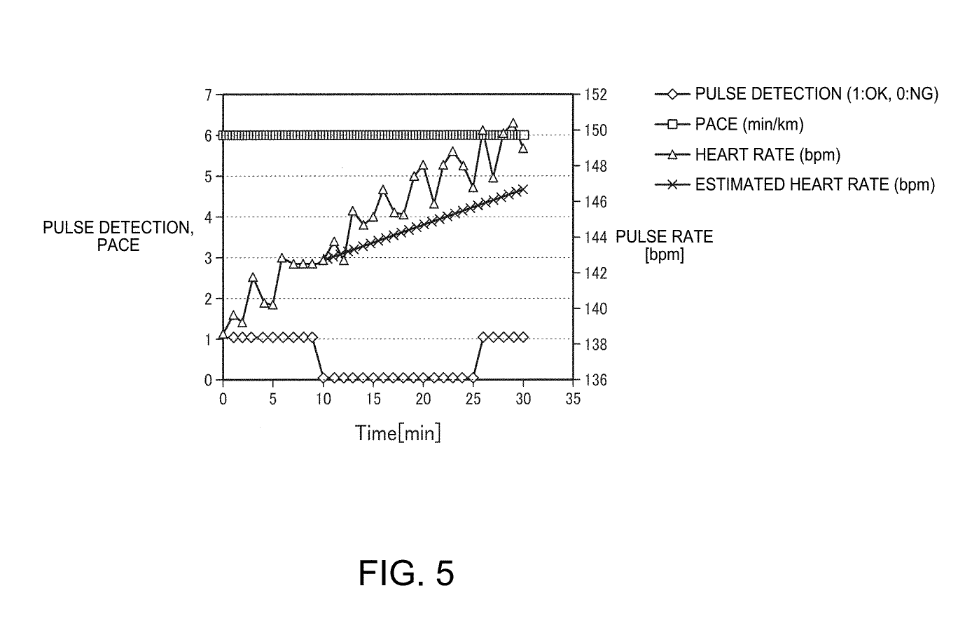

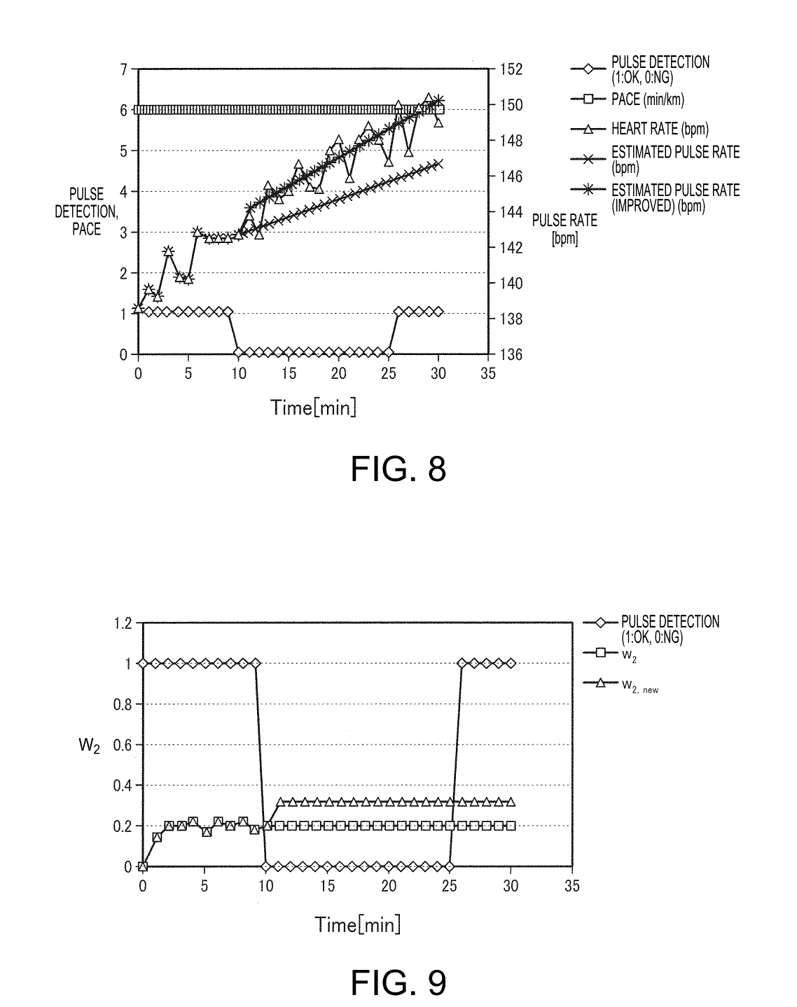

Hereinafter, specific examples will be described referring to FIGS. 5 to 9. As described above, if the time period during which the pulse rate cannot be measured becomes longer, attention needs to be paid to the phenomenon of heart rate drift. For example, the case shown in FIG. 5 where exercise with entirely the same activity intensity is continued is now considered. In the graph of FIG. 5, the horizontal axis represents the time elapsed (minutes. The vertical axis on the left-hand side of the graph of FIG. 5 represents both whether the pulse detection by the biological sensor 200 is OK (=1) or NG (=0) and the pace (moving speed) (min/km). The vertical axis on the right-hand side represents the pulse rate (bpm). The heart rate (bpm) is a value (measured value) measured by a heart rate sensor worn around the chest, as a reference. The same applies to the graph of FIG. 8, described later.

In the case of FIG. 5, the user continues running at the pace of 6 min/km for 30 minutes. During this time, the heart rate (measured value) starts at approximately 140 bpm and rises to approximately 150 bpm immediately before the end of the exercise. This is because the influence of the phenomenon of heart rate drift, in which the heart rate gradually increases even with the same exercise load (exercise with the same exercise intensity), emerges in the change in the pulse rate, as described above.

For example, in the case of FIG. 5, during the period from the elapsed time of 10 minutes to 25 minutes, the pulse detection is NG, that is, the pulse rate cannot be measured. Although the pulse detection becomes OK after 26 minutes, it is assumed that the learning of the coefficient information is not carried out when the pulse detection becomes OK again. That is, in the case of FIG. 5, from the start of the exercise up to the timing (before the lapse of 10 minutes) when the pulse rate can be still measured, the coefficient information is learned. However, after the measurement of the pulse rate is enabled again (after the lapse of 25 minutes), the coefficient information is not learned. In this case, if the heart rate is compared with the estimated heart rate after the end of the exercise (after the lapse of 30 minutes), the estimated heart rate is a lower value. Specifically, while the measured pulse rate is about 149 bpm, the estimated pulse rate is about 147 bpm. This is because the amount of increase in the pulse rate with the lapse of time is not correctly reflected. Particularly, if the period of learning becomes shorter, the number of samples decreases and the learning cannot take place sufficiently, as seen in this example. The samples (learning data) refer to case data including the measured pulse rate, the activity intensity at the time, and the activity time corresponding to the activity intensity, for example.

To prevent this, increasing the number of samples so as to increase the number of times the learning of the coefficient information takes place, is conceivable. However, if the pulse rate is NG due to low temperature, it is difficult to increase time brackets in which the pulse detection is OK, so as to increase the number of samples.

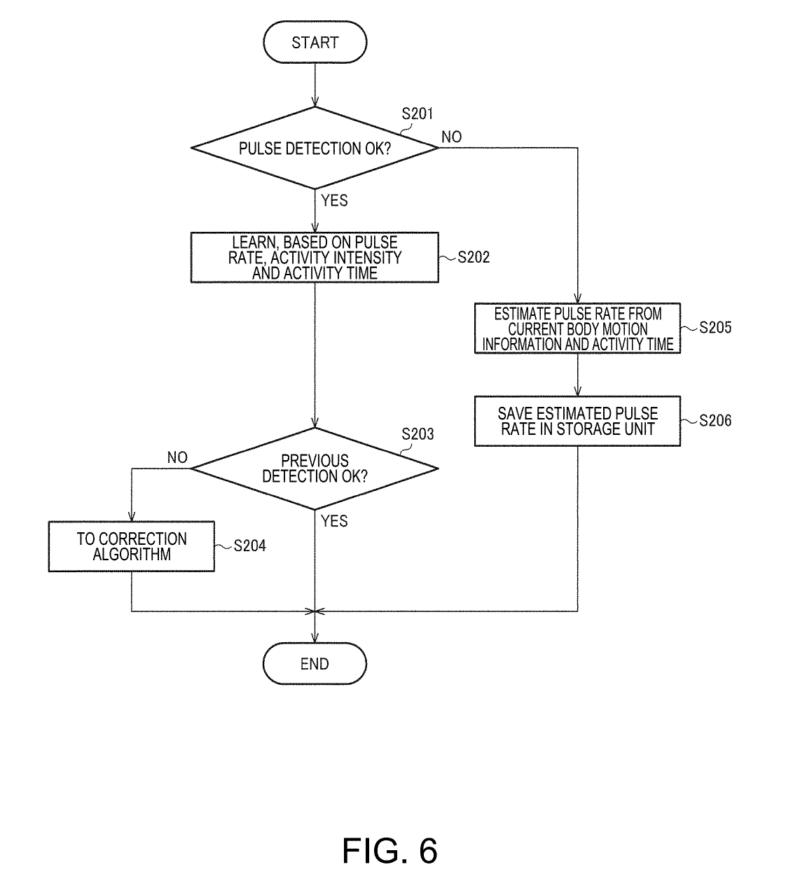

Thus, as in the example of FIG. 5, if the pulse detection becomes OK in the latter half, the coefficient information of the activity intensity and the activity time is updated and the past estimated pulse rate is thus corrected. A flow of processing in this case is shown in the flowchart of FIG. 6. Before Step S201 in FIG. 6, the processing of Steps S101 to S103 in FIG. 4 is carried out. The processing of Steps S201 to S206 in FIG. 6 is repeated on a predetermined cycle (for example, 10 seconds).

In the example shown in FIG. 6, the processing unit 130 determines whether the pulse rate is normally measured by the biological sensor 200 or not (S201), as in Step S104 in FIG. 4.

If the processing unit 130 determines that the pulse rate is normally measured (S201: YES), the processing unit 130 carries out learning using the detected pulse rate, the activity intensity and the activity time corresponding to the activity intensity, as in Step S105 in FIG. 4, and updates the coefficient information wₖ and wₖ₊ₘ in the equation (4), for example (S202). Next, the processing unit 130 determines whether the pulse rate is normally measured by the biological sensor 200 or not, at the time of the previous determination in Step 201 (S203). The time of the previous determination refers to the last time the series of processes in the flowchart of FIG. 6 is executed. If it is determined that the pulse rate is normally measured by the biological sensor 200 at the time of the previous determination in Step 201 (S203: YES), the processing ends there. That is, in this case, the pulse rate is normally measured by the biological sensor 200 both in the previous and current determinations. In the example shown in FIG. 5, this case corresponds to the period up to the timing when the pulse rate can no longer measured normally (period from 0 minutes to before the lapse of 10 minutes).

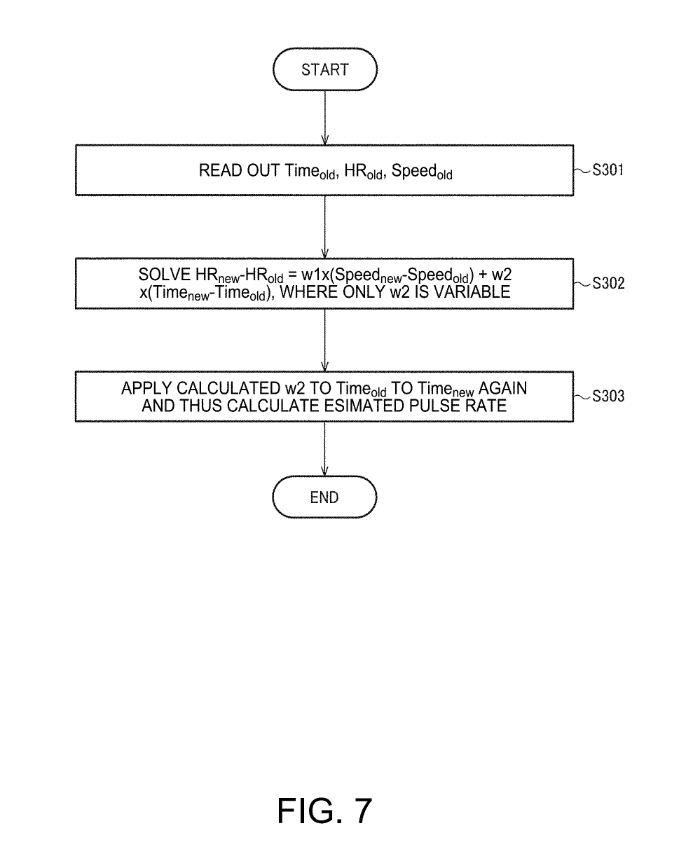

Meanwhile, if it is determined that the pulse rate cannot be measured normally by the biological sensor 200 at the time of the previous determination in Step S201 (S203: NO), the processing unit 130 executes a correction algorithm, described later with reference to FIG. 7, and updates the estimated pulse rate (S204). In this case, the ability to measure the pulse rate normally is restored from the state where the pulse rate cannot be measured normally. That is, in the example shown in FIG. 5, this case corresponds to the timing when 26 minutes have passed.

If it is determined that the pulse rate cannot be measured normally (S201: NO), the processing unit 130 estimates the pulse rate using the activity intensity and the activity time corresponding to the activity intensity (S205), as in Step S107 in FIG. 4. In the example shown in FIG. 5, for example, this case corresponds to the period from the time when 10 minutes have passed to the time when 25 minutes have passed. In this case, the estimated pulse rate “pulse” is found using the following equation (5), for example. The processing unit 130 then saves the estimated pulse rate in the storage unit 150 (S206) and ends the processing. In the equation below (5), “Speed” expresses the running pace (min/km) of the subject. As described above, in the example shown in FIG. 5, Speed=6 (min/km) holds. “Time” expresses the activity time (minutes) for which the activity is carried out at that pace (Speed). Also, w₁ is the coefficient information of the pace and w₂ is the coefficient information of the activity time.

pulse=pulseᵢₙᵢ+w₁×Speed+w₂×Time (5)

In this example, the indicator mainly indicating the phenomenon of heart rate drift is the coefficient information w₂ of the activity time. Therefore, if the ability to normally measure the pulse rate is restored from the state where the pulse rate cannot be measured normally (Step S204 as described above), the influence of the phenomenon of heart rate drift is reflected on the estimated pulse rate by updating the coefficient information w₂. A flow of processing of a correction algorithm carried out in this case is shown in the flowchart of FIG. 7.

First, the processing unit 130 reads out the time elapsed (Time_{old}) at the last time the pulse rate is normally measured, the pulse rate (HR_{old}) measured at the time, and the pace (Speed_{old}) at the time, from the storage unit 150 (S301). Next, the processing unit 130 solves the following equation (6), where only the coefficient information w₂ of the activity time is a variable (S302).

HR_{new}−HR_{old}=w₁×(Speed_{new}−Speed_{old})+w₂×(Time_{new}−Time_{old}) (6)

In the equation (6), Time_{new} is the time elapsed at the time when the pulse rate is successfully measured anew this time round. HR_{new} is the heart rate measured anew this time around. Speed_{new} is the pace at the time.

Next, the processing unit 130 applies the calculated coefficient information w₂ again to each timing during the period of Time_{old} to Time_{new} and thus calculates the estimated pulse rate during the period of Time_{old} to Time_{new}, using the equation (5) (S303). In the example shown in FIG. 5, the coefficient information w₂ is corrected based on the information stored during the period from the time elapsed of 10 minutes to 25 minutes, at the time point when 26 minutes have passed from the start of the exercise, and the estimated pulse rate is thus modified to the estimated pulse rate (improved) shown in FIG. 8. In the example shown in FIG. 8, the estimated pulse rate (improved) is increased according to the phenomenon of heart rate drift. The difference between the measured heart rate (measured value) and the estimated pulse rate (improved) at the time point when 30 minutes have passed is smaller than the difference between the heart rate (measured value and the original estimated pulse rate. Therefore, it can be said that the accuracy of estimation of the pulse rate is higher.

Thus, it is possible to acquire the estimated pulse rate with higher accuracy even in a time bracket in which the pulse rate cannot be measured directly. Changes in the coefficient information w₂ in this case are shown in FIG. 9. In the graph of FIG. 9, the horizontal axis represents the time elapsed (minutes). The vertical axis on the left-hand side represents the coefficient information w₂. The vertical axis on the right-hand side represents whether the pulse detection is OK (=1) or NG (=0). Also, W_{2,new} represents the coefficient information corrected by learning.

To summarize the above modification, the information acquisition unit 110 acquires first period biological information and a first period activity time before a non-detection period in which biological information cannot be acquired from the biological sensor 200, and acquires second period biological information and a second period activity time after the non-detection period. In the example shown in FIG. 5, for example, the non-detection period is the period from the time when 10 minutes have passed to the time when 25 minutes have passed. The first period biological information is the biological information acquired in the period preceding the non-detection period. The first period activity time is the activity time of the activity carried out by the subject during the period preceding the non-detection period. In example of the equation (6), for example, the first period biological information is HR_{old} and the first period activity time is Time old. The second period biological information is the biological information acquired in the period following the non-detection period. The second period activity time is the activity time of the activity carried out by the subject during the period following the non-detection period. In the example of the equation (6), for example, the second period biological information is HR_{new} and the second period activity time is Time_{new}. The processing unit 130 estimates the coefficient information, based on the first period biological information, the second period biological information, the first period activity time, and the second period activity time.

Thus, it is possible to reflect, on the estimated pulse rate, the influence of the phenomenon of heart rate drift that can occur during the biological information non-detection period, or the like.

Next, another modification will be described. The pulse rate does not immediately return to the normal value even if the subject who is exercising interrupts the exercise. It is often determined that the subject has more stamina as if takes longer for the pulse rate to return to the normal value. Also, depending on the exercise load, the pulse rate may not return to the normal value and may show a relatively high numerical value, even if a certain time has passed from the interruption of the exercise.

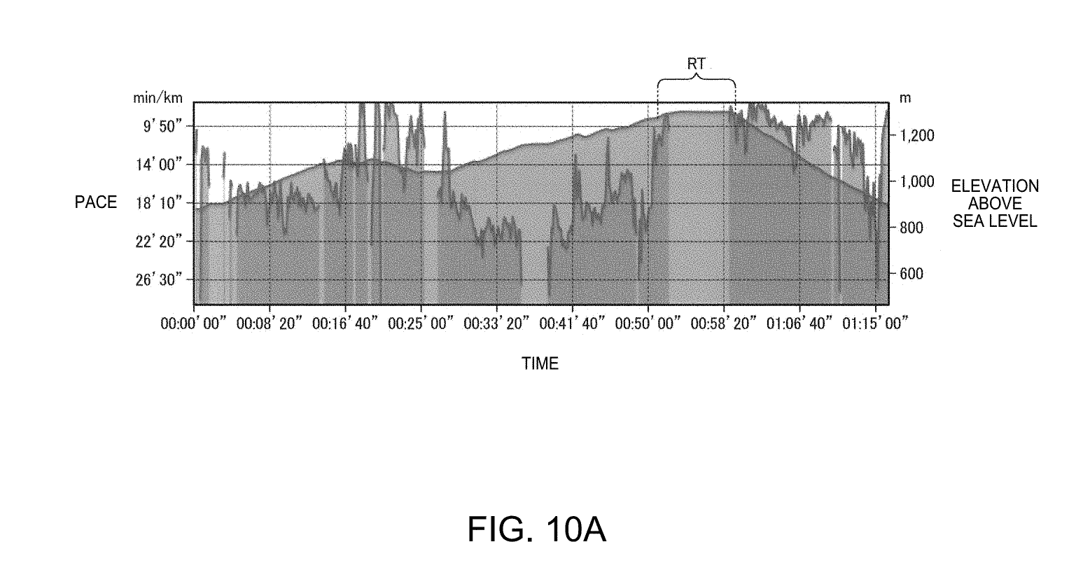

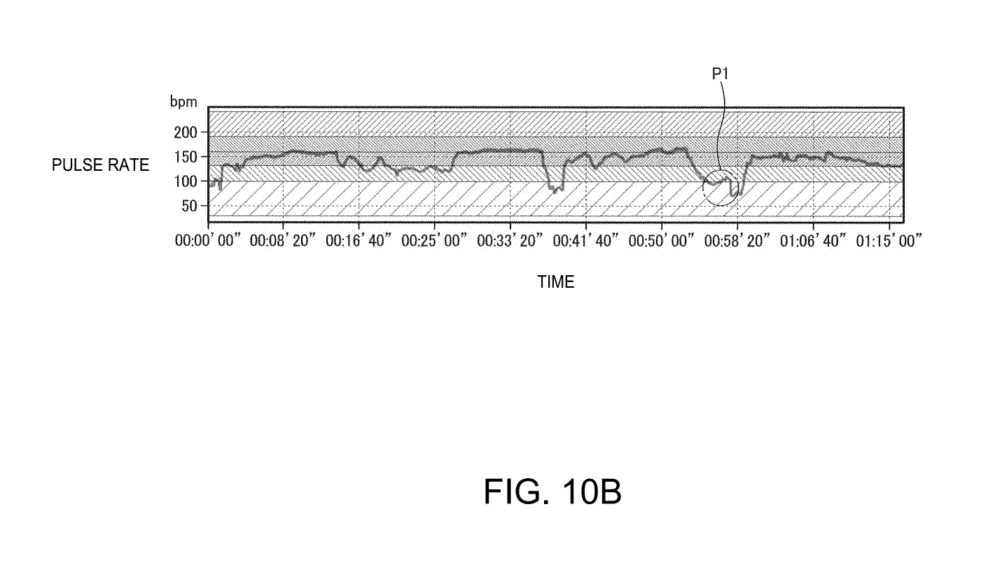

For example, the graph of FIG. 10A shows an example of the relation between the moving pace of the subject in mountaineering, the elevation above sea level, and the time elapsed. In the graph of FIG. 10A, the horizontal axis represents the time elapsed from the start of the exercise. The vertical axis on the left-hand side represents the moving pace (min/km). The vertical axis on the right-hand side represents the elevation above sea level (m) at the point where the subject is located. The graph of FIG. 10B shows the relation between the pulse rate and the time elapsed at the time when the subject carries out the activity shown in the graph of FIG. 10A. In the graph of FIG. 10B, the horizontal axis represents the time elapsed from the start of the exercise. The vertical axis represents the pulse rate (bpm). The time elapsed in the graph of FIG. 10A and the time elapsed in the graph of FIG. 10B are the same.

In the graph of FIG. 10A, the subject rests in a period indicated by RT. However, in FIG. 10B, though the subject stops the exercise at 52′32″, the pulse rate of normal time (here, 55 to 60) is not restored in a period indicated by P1.

Thus, in this modification, when it is determined that the pulse rate is stabilized after it is determined that the exercise is stopped, the degree of consumption of stamina of the subject is found using the pulse rate at the time (for example, 98 in the example shown in FIG. 10B). Then, the estimated pulse rate is corrected, based on the degree of consumption of stamina.

That is, in this modification, the information acquisition unit 110 acquires post-activity biological information from the biological sensor 200 after the lapse of a predetermined period following the stop of the activity of the subject. The processing unit 130 then specifies stamina information indicating the stamina consumed by the subject by carrying out the activity, based on the post-activity biological information, and estimates biological information based on the stamina information.

Specifically, in this modification, the coefficient information w₂ of the activity time in the equation (5) corresponds to the stamina information indicating the stamina consumed by the subject. In this modification, in order to find the stamina information based on the pulse rate at the time when it is determined that the pulse rate is stabilized when the activity is stopped, the coefficient information w₂ is updated to w_{2,new} by the following equation (7). In the equation (7), pulse_{break} is the pulse rate when it is determined that the pulse rate is stabilized when the activity is stopped. In the example shown in FIG. 10B, for example, pulse_{break}=98 holds. “Time” in the equation (7) is the time from the start of the exercise to the rest. In the example shown in FIG. 10B, for example, this time is 52′32″.

$${w}_{2 , new} = \frac{{pulse}_{break} - {pulse}_{int}}{Time} \qquad ( 7 )$$

The resulting coefficient information w_{2,new} is substituted for w₂ in the equation (5), thus updating the estimated pulse rate.

Thus, by updating the estimated pulse rate of the subject, based on the stamina information of the subject, it is possible to improve the accuracy of estimation of the pulse rate, or the like.

The processing unit 130 may generate notification information of the stamina information, based on the coefficient information corresponding to the activity time.

Thus, it is possible to display the amount of stamina left as the stamina information, for example, on a display unit, not illustrated. Therefore, the subject can carry out the activity while checking his/her own stamina information, or the like.

Next, still another modification will be described. In this modification, the processing unit 130 carries out action determination processing in which the action of the subject is determined, based on body motion information, and changes coefficient information, based on the result of the action determination processing.

In this case, for example, if the processing unit 130 determines that the action of the subject is a first action, the processing unit 130 estimates a first type activity time when the subject carries out the first action, and first type time coefficient information corresponding to the first type activity time, and estimates first biological information based on the first type activity time and the first type time coefficient information. Meanwhile, if the processing unit 130 determines that the action of the subject is a second action that is different from the first action, the processing unit 130 estimates a second type activity time when the subject carries out the second action, and second type time coefficient information corresponding to the second type activity time, and estimates second biological information based on the second type activity time and the second type time coefficient information.

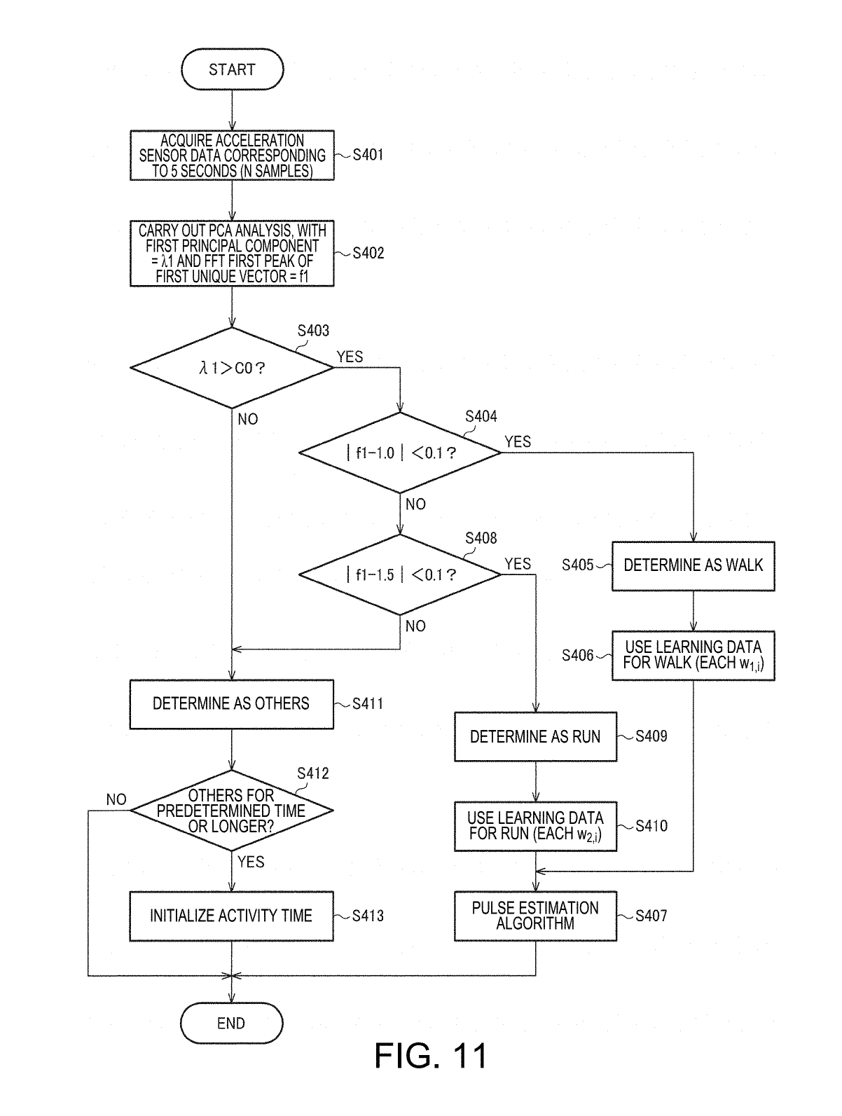

A specific example is shown in the flowchart of FIG. 11. First, the information acquisition unit 110 acquires acceleration sensor data corresponding to 5 seconds (N samples), for example, from an acceleration sensor as the body motion sensor 300 (S401). The processing unit 130 then carries out PCA analysis (principal component analysis) based on the acquired acceleration sensor data (S402). Here, a first principal component in the PCA analysis is referred to as λ1, and an FFT first peak of a first unique vector is referred to as f1.

The first principal component λ1 corresponds to the amount of change in acceleration. Therefore, the processing unit 130 determines whether the value of λ1 is greater than a predetermined threshold C0 or not (S403). If it is determined that λ1>C0 holds (S403: YES), it is determined that the subject is exercising.

The FFT first peak f1 of the first unique vector corresponds to the frequency of change in acceleration. Therefore, if it is determined that the subject is exercising, it is determined that the subject is walking when the value of f1 is approximately 1.0 Hz, and that the subject is running when the value of f1 is approximately 1.5 Hz. In this modification, such processing is carried out by determining whether the difference between the value of f1 and a target value is greater than a predetermined allowable difference range or not. Here, the predetermined allowable difference range is 0.1 Hz, for example. As described above, the target value is 1.0 Hz in the case of walking and 1.5 Hz in the case of running. That is, specifically, the processing unit 130 determines whether |f1−1.0| is smaller than 0.1 Hz or not (S404). If it is determined that |f1−1.0| is smaller than 0.1 Hz (S404: YES), it is determined that the subject is walking (S405). At this time, walking (Walk) is defined as the first action. The processing unit 130 uses learning data (each coefficient information w_{1,i}) for Walk (S406), executes a pulse estimation algorithm (S407), and thus finds an estimated pulse rate for Walk. In this case, the first type activity time is the time taken by the subject for walking. The first type time coefficient information is w_{1,i}. The first biological information is the estimated pulse rate for Walk. The pulse estimation algorithm is the processing shown in FIG. 4, the equation (4) and the like. The value of w_{1,i} is used for wₖ and wₖ₊ₘ in the equation (4).

Meanwhile, if it is determined that |f1−1.0| is 0.1 Hz or greater (S404: NO), it is then determined that |f1−1.5| is smaller than 0.1 Hz or not (S408). If it is determined that |f1−1.5| is smaller than 0.1 Hz (S408: YES), it is determined that the subject is running (S409). At this time, running (Run) is defined as the second action. The processing unit 130 uses learning data (each coefficient information w_{2,i}) for Run (S410), executes the pulse estimation algorithm (S407), and finds an estimated pulse rate for Run. In this case, the second type activity time is the time taken by the subject for running. The second type time coefficient information is w_{2,i}. The second biological information is the estimated pulse rate for Run.

If it is determined that λ1≦C0 (S403: NO), it is determined that the subject is not exercising (S411). Also, if it is determined that |f1−1.5| is 0.1 Hz or greater (S408: NO), it is determined that the action of the subject is neither walking nor running (S411). In these cases, the processing unit 130 determines whether or not the time during which the subject neither walks nor runs is a predetermined time or longer (S412). If it is determined that the time during which the subject neither walks nor runs is a predetermined time or longer (S412: YES), the phenomenon of heart rate drift is not considered to have occurred and therefore the processing of initializing (resetting) the activity time of the subject is carried out (S413). That is, in the action determination processing, if the processing unit 130 determines that the subjects is in a stopped state, for example, the processing unit 130 carries out the processing of initializing the activity time. Specifically, the activity time of the subject that is stored temporarily in the storage unit 150 for the use in learning the coefficient information is erased.

If it is determined that the time during which the subject neither walks nor runs is shorter than a predetermined time (S412: NO), the processing ends and the processing shown in FIG. 11 is executed again at the next determination timing.

Thus, it is possible to switch the coefficient information to be learned, according to the action of the subject, and estimate the pulse rate corresponding to the action of the subject, or the like.

Also, it is possible to improve the accuracy of estimation of the first biological information at the time when the subject carries out the first action, and also improve the accuracy of estimation of the second biological information at the time when the subject carries out the second action, or the like.

Moreover, if it is presumed that the phenomenon of heart rate drift has not occurred, it is possible to estimate the pulse rate without considering the influence of the phenomenon of heart rate drift, or the like. Thus, it is possible to improve not only the accuracy of estimation of biological information in the case where the phenomenon of heart rate drift occurs but also the accuracy of estimation of biological information in the case where the phenomenon of heart rate drift does not occur, or the like.

In mountaineering, for example, the subject often continuously carries out a plurality of types of activities such as climbing, descending, and moving on flat land, in one session of mountaineering. In the case where the subject checks the pulse rate during such mountaineering, a pulse rate estimated in consideration of all of these actions (climbing, descending, and the like) is more useful than estimated pulse rates corresponding to the respective actions.

Therefore, in this modification, for example, if the processing unit 130 determines that the action of the subject is a first action, the processing unit 130 may estimate a first type activity time at the time when the subject carries out the first action, and first type time coefficient information corresponding to the first type activity time. Meanwhile, if the processing unit 130 determines that the action of the subject is a second action that is different from the first action, the processing unit 130 may estimate second type activity time at the time when the subject carries out the second action, and second type time coefficient information corresponding to the second type activity time. The processing unit 130 may then estimate biological information, based on the first type activity time information, the first type time coefficient information, the second type activity time, and the second type time coefficient information.

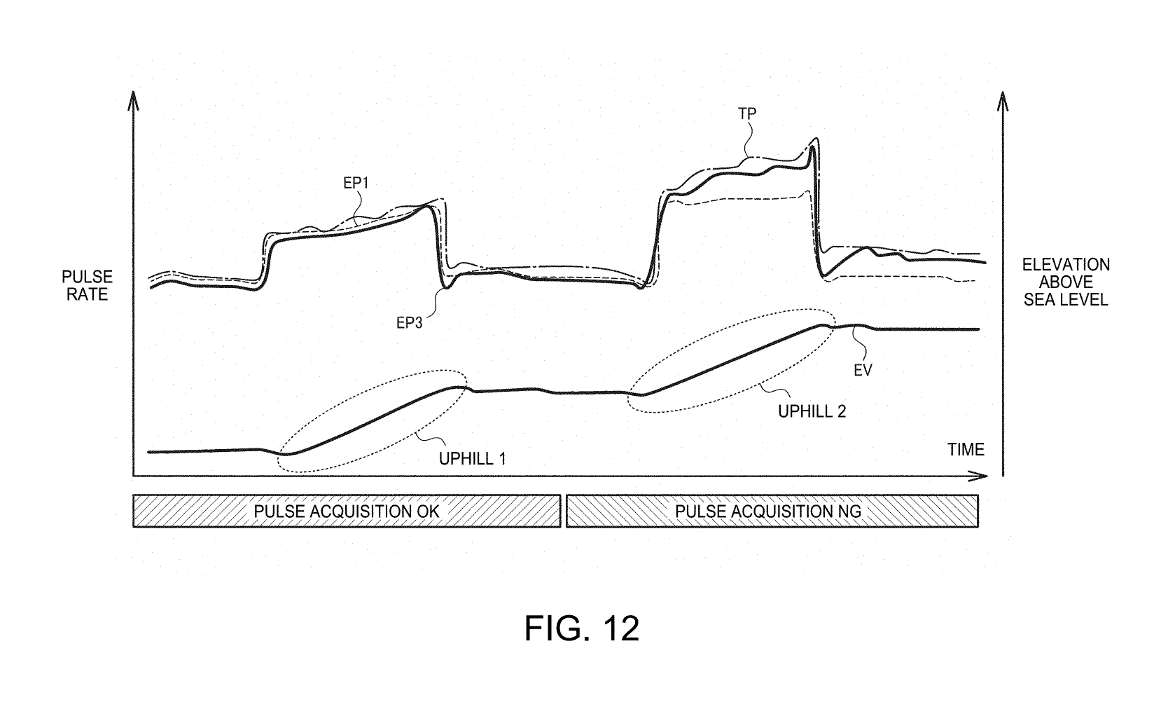

A specific example is shown in FIG. 12. The graph of FIG. 12 shows the relation between the pulse rate of the subject in mountaineering, the elevation above sea level, and time. In the graph of FIG. 12, the horizontal axis represents time. The vertical axis on the left-hand side represents the pulse rate. The vertical axis on the right-hand side represents the elevation above sea level. In the example shown in FIG. 12, it is assumed that the subject moves at roughly the same pace. In this example, as can be seen from the time elapsed corresponding to the elevation above sea level EV, the subject moves on a road including flat land and two uphills (uphill 1 and uphill 2), and TP indicates the true value of the pulse rate at the time. As shown in the graph of FIG. 12, the pulse rate TP is higher on the uphills than on the flat land, and the degree of rise in the pulse rate per unit time on the uphills tends to be higher. The pulse rate can be measured by the biological sensor 200 until around the end of going up the uphill 1. However, the pulse rate can no longer be measured after that.

In this case, after the pulse rate can no longer be measured, the influence of the phenomenon of heart rate drift cannot be reflected well on the estimated pulse rate even if the coefficient information w₂ in the equation (5) or the like is learned, using only the cumulative activity time from the start of climbing. Specifically, the physical strength consumed on the uphill 2 cannot be taken into account when the pulse rate is ultimately estimated. Therefore, as indicated by EP1 in FIG. 12, the estimated pulse rate is calculated to be lower than the actual pulse rate after the pulse rate can no longer be measured.

Thus, in this modification, an activity time (first type activity time) Ta when the subject moves on flat land, an activity time (second type activity time) Tb when the subject moves on an uphill, and an activity time (third type activity time) Tc when the subject moves on a downhill are used. Then, coefficient information (first type time coefficient information) w_{2,a} corresponding to the activity time Ta when the subject moves on the flat land, coefficient information (second type time coefficient information) w_{2,b} corresponding to the activity time Tb when the subject moves on the uphill, and coefficient information (third type time coefficient information) w_{2,c} corresponding to the activity time Tc when the subject moves on the downhill are learned and estimated. Then, the estimated pulse rate “pulse” is found by the following equation (8).

pulse=pulseᵢₙᵢ+w₁×Speed+w_{2,a}×Ta+w_{2,b}×Tb+w_{2,c}×Tc (8)

In the graph of FIG. 12, the estimated pulse rate found by the equation (8) is indicated by EP3. In this case, as indicated by EP3, it is possible to reflect the influence of the consumption of physical strength on the uphill 2, on the estimated pulse rate, and thus make the estimated pulse rate closer to the true value.

As described above, in the case where the pulse rate cannot be measured by the biological sensor, it is possible to estimate the pulse rate more accurately, or the like, even if the subject carries out a plurality of types of activities together.

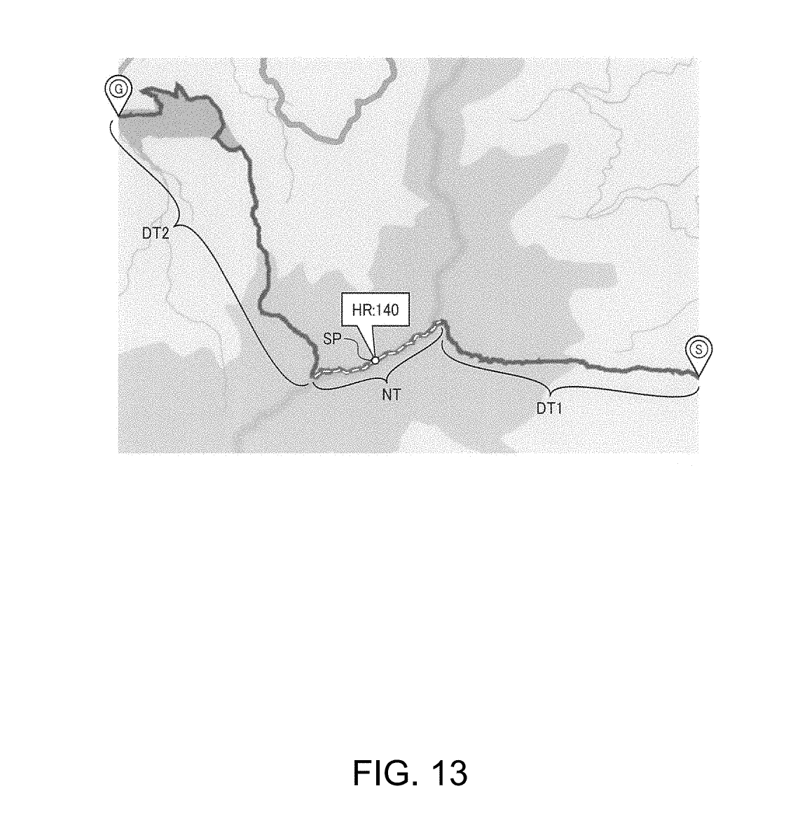

In the case of mountaineering as shown in FIG. 12, for example, the transition of pulse rate may be checked after the mountaineering. In this case, the section where the pulse rate cannot be measured by the biological sensor 200 may be displayed on a map, along with the traveling route of the subject, as shown in FIG. 13, for example. The example shown in FIG. 13 shows that the pulse rate is successfully measured in sections DT1 and DT2 and that the pulse rate cannot be measured in a section NT. On the map, the pulse rate at a point selected by the user is displayed as well. In the example shown in FIG. 13, for example, the pulse rate HR at a point SP in the section NT is displayed as 140.

In this case, if the information acquisition unit 110 cannot acquire biological information from the biological sensor 200, the processing unit 130 generates non-detection period notification information indicating the period in which biological information cannot be acquired. The processing unit 130 then displays the non-detection period notification information on a display unit, not illustrated. The non-detection period notification information is image information showing the traveling route of the subject in the biological information non-detection period, character information representing the pulse rate, or icon information or the like. In the example shown in FIG. 13, for example, the non-detection period notification information is information for changing the color of the section NT or the estimated pulse rate or for switching on and off the display of the section NT or the estimated pulse rate.

Thus, it is possible to notify the subject (user) of the biological information non-detection period, or the like. The user can, for example, grasp whether the displayed pulse rate is a measured value or estimated value. Also, in the example shown in FIG. 13, for example, it can be understood that the pulse rate cannot be measured by the biological sensor 200 only while the subject is moving on a ridgeline. Therefore, the cause of the inability to measure the pulse rate can be analyzed, such as a drop in the temperature of the device due to the influence of wind or the like. Particularly when the pulse detection cannot be carried out because of low temperature, the area in which the temperature of the device drops can be grasped and this information can be used to determine how to estimate the accuracy of the recorded pulse rate. Particularly when the pulse detection is unavailable only on the ridgeline of the mountain where there is strong wind, measures to increase the detection rate in the subsequent sessions can be taken, such as covering the device with a windproof material.

Immediately after the subject starts exercise, the pulse rate may not correspond to the actual exercise load (activity intensity). This is because the pulse rate does not become stable until an activity is continued for a certain period of time. Moreover, the time taken for the pulse rate to become stable varies from one individual to another. Therefore, it is better to carry out the pulse rate estimation processing shown in FIG. 4 or the like, after it is determined that the activity of the subject is in a stable state.

In this case, the information acquisition unit 110 acquires body motion information from the body motion sensor 300 and acquires biological information from the biological sensor 200. The processing unit 130 determines whether the activity of the subject is in a stable state or not, based on the body motion information and the biological information, and estimates biological information based on the activity time after it is determined that the activity of the subject is in a stable state.

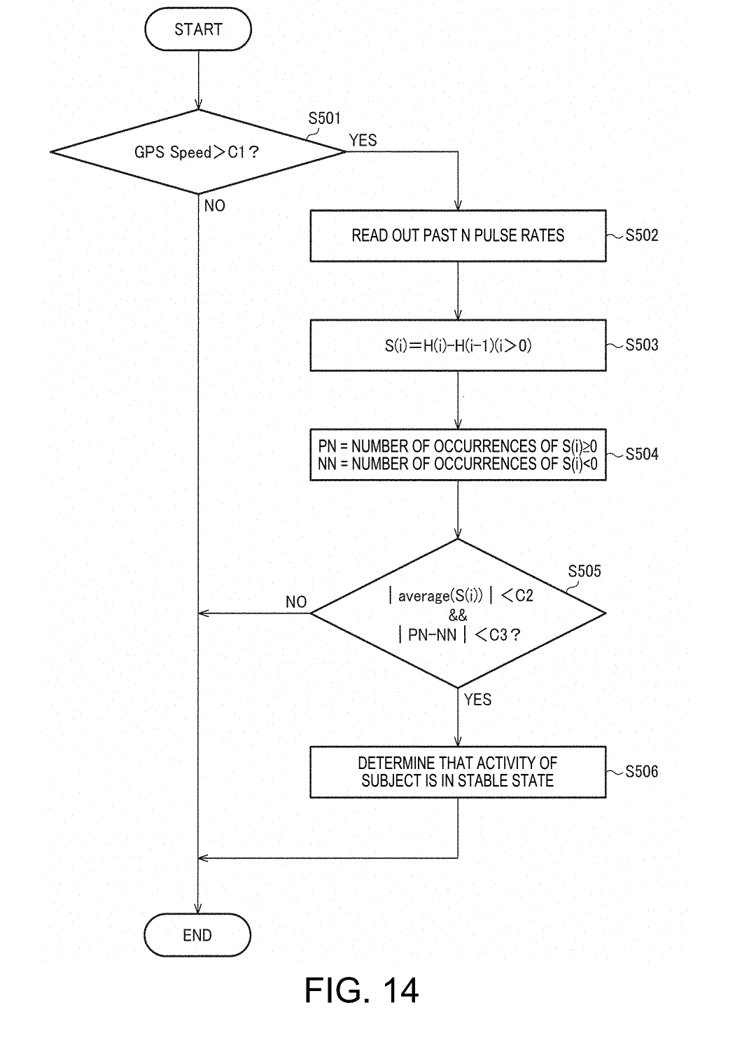

A specific flow of determination processing is shown in FIG. 14. In the example shown in FIG. 14, it is determined that the activity of the subject is in a stable state, if the amount of change in the pulse rate between measurement timings next to each other is smaller than a certain threshold and the pulse rate is neither in an upward trend nor in a downward trend.

First, in the example shown in FIG. 14, a GPS receiver is used as a body motion sensor, and the information acquisition unit 110 acquires a GPS receiving signal. The processing unit 130 determines whether the moving pace (GPS Speed) of the subject calculated based on the GPS receiving signal is greater than a predetermined threshold C1 or not (S501).

If the processing unit 130 determines that the moving pace (GPS Speed) of the subject is greater than the predetermined threshold C1, the processing unit 130 determines that a certain period has passed from the start of the activity, and reads out past N pulse rates from the storage unit 150 (S502). Here, the pulse rates that are read out are expressed by H(i), where i is a variable indicating the measuring order.

The processing unit 130 then calculates the difference S(i) between two pulse rates measured at measuring timings next to each other, by S(i)=H(i)−H(i−1) (S503). Next, the processing unit 130 calculates the number PN of occurrences of S(i) equal to 0 or greater and the number NN of occurrences of S(i) smaller than 0 (S504). S(i) equal to 0 or greater means that the pulse rate is increased. PN corresponds to the number of samples where pulse rates measured at measuring timings next to each other show an increase. Similarly, S(i) being smaller than 0 means that the pulse rate is lowered. NN corresponds to the number of samples where pulse rates measured at measuring timings next to each other show a decrease. The processing unit 130 also determines whether |average(S(i))|<C2 and |PN−NN|<C3 hold or not (S505). Average(S(i)) is an average value of the S(i) calculated in S503. C2 and C3 are predetermined thresholds. That is, here, the processing unit 130 determines whether the average value |average(S(i))| of the difference S(i) of two pulse rates measured at measuring timings next to each other is smaller than the predetermined threshold C2 or not, and whether the difference |PN−NN| between the number PN of increases between pulse rates measured at measuring timings next to each other and the number NN of decreases is smaller than the predetermined threshold C3 or not, as described above. If the processing unit 130 determines that |average(S(i))|<C2 and |PN−NN|<C3 hold (S505: YES), the processing unit 130 determines that the activity of the subject is in a stable state (S506) and subsequently carries out the processing shown in FIG. 4 or the like, for example.

Meanwhile, if the processing unit 130 determines that the moving pace (GPS Speed) of the subject is equal to the predetermined threshold C1 or below, or that |average(S(i))|<C2 and |PN−NN|<C3 do not hold (S505: NO), the processing unit 130 determines that the activity is not started or that the pulse rate is not stable because the activity is started shortly before. The processing unit 130 then ends the processing.

Thus, it is possible to estimate the pulse rate after it is determined that the activity of the subject is in a stable state, and to improve the accuracy of the estimated pulse rate, or the like.

### 4. Specific Example of Wearable Device

FIGS. 15A to 15C show an example of the appearance of the wearable device 500 (wearable apparatus) acquiring biological information and body motion information. The wearable device 500 in this embodiment has a strap part 10, a case part 30, and a sensor part 40. As shown in FIGS. 15A and 15B, the case part 30 is installed on the strap part 10. As shown in FIG. 15C, the sensor part 40 is provided in the case part 30 and includes the biological sensor 200 and the body motion sensor 300. The sensor part 40 includes the biological sensor 200 and the body motion sensor 300 shown in FIG. 1.

The strap part 10 is to be wound around the wrist of the user so that the user can wear the wearable device 500. The strap part 10 has a strap hole 12 and a buckle part 14. The buckle part 14 has a strap insertion part 15 and a protruding part 16. The user inserts one end of the strap part 10 into the strap insertion part 15 of the buckle part 14 and inserts the protruding part 16 of the buckle part 14 into the strap hole 12 of the strap part 10, thus wearing the wearable device 500 around the wrist. The strap part 10 may have a clasp instead of the buckle part 14.

The case part 30 corresponds to the main body part of the wearable device 500. Inside the case part 30, various components of the wearable device 500 such as the sensor part 40 and a circuit board, not illustrated. That is, the case part 30 is a casing accommodating these components.

A light emitting window part 32 is provided in the case part 30. The light emitting window part 32 is formed of a light-transmitting member. A light emitting unit as an interface mounted on a flexible substrate is provided in the case part 30, and the light from the light emitting unit is emitted out of the case part 30 via the light emitting window part 32. Also, in the case part 30, a display unit such as an LCD (Liquid Crystal Display) may be provided instead of the light emitting unit, or the display unit and the light emitting unit may be provided together.

The wearable device 500 is worn around the wrist of the user, as shown in FIG. 16A or the like. In this wearing state, biological information and body motion information are measured.

### 5. Specific Example of Implementation of Biological Information Processing System

Next, a specific example of a device which implements the biological information processing system 100 according to the embodiment will be described. The functions of the biological information processing system 100 may be implemented by a server system 600, for example. An example of this case is FIG. 16A. For example, the biological information processing system 100, which is the server system 600, is connected to a wearable device 500 (electronic device) via a network NE and acquires biological information and body motion information of the subject from this wearable device 500. Since the wearable device 500 worn by the user needs to be small-sized and lightweight, the processing capability of the battery and the processing unit inside the device, or the data storage capacity is greatly limited. Meanwhile, the server system 600 has less limitation to its resources and therefore can carryout the processing of estimating biological information based on body motion information and can hold more data (biological history information and body motion history information or the like), for example.

It suffices that the biological information processing system 100 can acquire various types of information collected by the wearable device 500. Therefore, the biological information processing system 100 is not limited to being directly connected to the wearable device 500. For example, as shown in FIG. 16B, the wearable device 500 may be connected to another processing device 700, and the biological information processing system 100 may be connected to the processing device 700 via a network NE. The processing device 700 in this case may be a portable terminal device such as a smartphone used by the user wearing the wearable device 500, for example. For the connection between the wearable device 500 and the processing device 700, a measure similar to the network NE may be used and short-range wireless communication or the like can be used as well.

The biological information processing system 100 according to the embodiment may also be implemented by the processing device 700 (electronic device; in a narrow sense, portable terminal device) such as a smartphone, instead of the server system 600. An example of the configuration in this case is FIG. 16C. The portable terminal device such as a smartphone often has more limitations to the processing capability, storage area and battery capacity than the server system 600. However, given the recent improvement in capabilities, the portable terminal device may be able to secure sufficient processing capability. Therefore, if the requirement of processing capability or the like is satisfied, a smartphone or the like can be used as the biological information processing system 100 according to the embodiment, as shown in FIG. 16C.

Moreover, when the improvement in the capabilities or the use of the terminal, a form of embodiment in which the wearable device 500 (electronic device) includes the biological information processing system 100 according to the embodiment as described above may be employed. In this case, the information acquisition unit 110 acquires information from the biological sensor 200 and the body motion sensor 300 provided inside the same device. In the case where the biological information processing system 100 is installed in the wearable device 500, the biological information processing system 100 is less likely to need to perform data analysis, saving or the like for a large number of users, and may only have to target one or a small number of users using the wearable device 500. That is, it is very likely that even the processing capability of the wearable device 500 can satisfy the needs of the user.

That is, the method in the embodiment can be applied to a terminal device (biological information processing device, biological information analysis device, biological information measuring device, biological information detection device) including an information acquisition unit which acquires body motion information of the subject and a processing unit which estimates biological information based on the acquired body motion information.

In the above description, the biological information processing system 100 is implemented by one of the server system 600, the processing device 700, and the wearable device 500. However, this is not limiting. For example, the processing of acquiring body motion information and the processing of estimating biological information may be implemented by distributed processing by a plurality of devices. Specifically, the biological information processing system 100 may be implemented by at least two or more of the server system 600, the processing device 700, and the wearable device 500. Alternatively, another device may perform a part of the processing by the biological information processing system 100. The biological information processing system 100 according to the embodiment can be implemented by various devices (or combinations of devices).

In the biological information processing system and the electronic device or the like according to the embodiment, a part or a majority of the processing may be implemented by a program. In this case, a processor such as a CPU executes the program, thus implementing the biological information processing system and the electronic device or the like according to the embodiment. Specifically, the program stored in a non-temporary information storage device is read out, and the processor such as a CPU executed the read-out program. Here, the information storage device (computer-readable device) stores a program, data and the like. The functions of the information storage device can be implemented by an optical disk (DVD, CD or the like), HDD (hard disk drive), or memory (memory card, ROM or the like). The processor such as a CPU carries out various kinds of processing in the embodiment, based on the program (data) stored in the information storage device. That is, a program which causes a computer (device having an operation unit, a processing unit, a storage unit, and an output unit) to function as each component of the embodiment (program for causing a computer to execute the processing by each component) is stored in the information storage device.

Thus, the processing in the embodiment can be implemented by a program. The program may be, for example, a program read out and executed by the processing unit of a device like a smartphone (for example, DSP).

Although the embodiment has been described in detail above, a person skilled in the art can readily understand that various modifications can be made without substantially departing from the new matters and advantageous effects of the invention. Therefore, all such modifications are included in the scope of the invention. For example, a term described along with a different term with a broader meaning or the same meaning at least once in the specification or drawings can be replaced by the different term at any point in the specification or drawings. Also, the configurations and operations of the biological information processing device and the program are not limited to those described in the embodiment and can be carried out with various modifications.

The entire disclosure of Japanese Patent Application No. 2016-049633, filed Mar. 14, 2016 is expressly incorporated by reference herein.

What is claimed is:

1. A biological information processing system comprising:
a body motion sensor;
an information acquisition unit which acquires body motion information of a subject from the body motion sensor; and
a processing unit which specifies an activity intensity of the subject and an activity time corresponding to the activity intensity, based on the body motion information that is acquired, and estimates biological information of the subject, based on the activity time.

2. The biological information processing system according to claim 1, wherein
the processing unit estimates biological information, based on first to N-th activity times corresponding to first to N-th activity intensities of the subject, with N being an integer equal to or greater than 2.

3. The biological information processing system according to claim 1, further comprising
a biological sensor,
wherein, when the information acquisition unit cannot acquire the body motion information of the subject from the body motion sensor, the processing unit estimates biological information, based on the activity time corresponding to the activity intensity.

4. The biological information processing system according to claim 1, wherein
the processing unit estimates biological information, based on the activity time and coefficient information corresponding to the activity time.

5. The biological information processing system according to claim 4, wherein
the processing unit
estimates first to N-th coefficient information corresponding to the activity time, and
estimates biological information, based on the activity time and the coefficient information.

6. The biological information processing system according to claim 1, further comprising
a biological sensor,
wherein if the information acquisition unit can acquire biological information from the biological sensor, the processing unit estimates the coefficient information corresponding to the activity time, based on the biological information.

7. The biological information processing system according to claim 6, wherein
the information acquisition unit
acquires first period biological information and a first period activity time before a non-detection period during which the biological information cannot be acquired from the biological sensor, and
acquires second period biological information and a second period activity time after the non-detection period, and
the processing unit estimates the coefficient information corresponding to the first period biological information, the second period biological information, the first period activity time, and the second period activity time.

8. The biological information processing system according to claim 7, wherein
the processing unit
carries out action determination processing in which an action of the subject is determined based on the body motion information, and
changes the coefficient information, based on a result of the determination in the action determination processing.

9. The biological information processing system according to claim 8, wherein
if the processing unit determines that the action of the subject is a first action,
the processing unit
estimates a first type activity time during which the subject carries out the first action, and first type time coefficient information corresponding to the first type activity time, and
estimates first biological information, based on the first type activity time and the first type time coefficient information, and
if the processing unit determines that the action of the subject is a second action that is different from the first action,
the processing unit
estimates a second type activity time during which the subject carries out the second action, and second type time coefficient information corresponding to the second type activity time, and
estimates second biological information, based on the second type activity time and the second type time coefficient information.

10. The biological information processing system according to claim 8, wherein
the processing unit carries out processing of initializing the activity time if the subject is determined as being in a stopped state, in the action determination processing.

11. The biological information processing system according to claim 8, wherein
if the processing unit determines that the action of the subject is a first action,
the processing unit estimates a first type activity time during which the subject carries out the first action, and first type time coefficient information corresponding to the first type activity time, and
if the processing unit determined that the action of the subject is a second action that is different from the first action,
the processing unit estimates a second type activity time during which the subject carries out the second action, and second type time coefficient information corresponding to the second type activity time, and
the processing unit estimates biological information, based on the first type activity time, the first type time coefficient information, the second type activity time, and the second type time coefficient information.

12. The biological information processing system according to claim 3, wherein
the information acquisition unit acquires post-activity biological information from the biological sensor after the lapse of a predetermined period following the stop of the activity of the subject, and
the processing unit
specifies stamina information indicating stamina consumed by the subject in carrying out the activity, based on the post-activity biological information, and
estimates biological information, based on the stamina information.

13. The biological information processing system according to claim 12, wherein
the processing unit generates notification information of the stamina information, based on coefficient information corresponding to the activity time.

14. The biological information processing system according to claim 1, wherein
the processing unit
determines whether an activity of the subject is in a stable state or not, based on the body motion information and the biological information, and
estimates biological information, based on the activity time after it is determined that the activity of the subject is in the stable state.

15. The biological information processing system according to claim 3, wherein
the processing unit generates non-detection period notification information indicating a period during which the biological information cannot be acquired.

16. The biological information processing system according to claim 1, wherein
the biological information processing system is a wearable device.

17. The biological information processing system according to claim 1, comprising:
a wearable device; and
a server;
wherein the wearable device includes the body motion sensor, and
the server includes the information acquisition unit and the processing unit.

18. The biological information processing system according to claim 1, comprising:
a wearable device; and
a portable terminal device;
wherein the wearable device includes the body motion sensor, and
the portable terminal device includes the information acquisition unit and the processing unit.

19. A server comprising:
an information acquisition unit which acquires body motion information of a subject from a body motion sensor; and
a processing unit which specifies an activity intensity of the subject and an activity time corresponding to the activity intensity, based on the body motion information that is acquired, and estimates biological information of the subject, based on the activity time.

20. The server according to claim 19, wherein
the processing unit estimates biological information, based on first to N-th activity times corresponding to first to N-th activity intensities of the subject, with N being an integer equal to or greater than 2.

21. A biological information estimation method comprising:
acquiring body motion information of a subject from a body motion sensor;
specifying an activity intensity of the subject and an activity time corresponding to the activity intensity, based on the body motion information; and
estimating biological information of the subject, based on the activity time.

22. The biological information estimation method according to claim 21, wherein
the estimating includes
estimating biological information, based on first to N-th activity times corresponding to first to N-th activity intensities of the subject, with N being an integer equal to or greater than 2.

23. The biological information estimation method according to claim 22, wherein
the estimating includes
estimating biological information, based on the activity time and coefficient information corresponding to the activity time.

24. The biological information estimation method according to claim 23, wherein
the estimating includes
estimating first to N-th coefficient information corresponding to the activity time, and
estimates biological information, based on the activity time and the coefficient information.

25. A wearable device which
acquires body motion information of a subject from a body motion sensor,
specifies an activity intensity of the subject and an activity time corresponding to the activity intensity, based on the body motion information that is acquired, and
estimates biological information of the subject, based on the activity time.

26. The wearable device according to claim 25, wherein
the estimation includes
estimating biological information, based on first to N-th activity times corresponding to first to N-th activity intensities of the subject, with N being an integer equal to or greater than 2.

27. The wearable device according to claim 26, wherein
the estimation includes
estimating biological information, based on the activity time and coefficient information corresponding to the activity time.

28. The wearable device according to claim 27, wherein
the estimation includes
estimating first to N-th coefficient information corresponding to the activity time, and
estimating biological information, based on the activity time and the coefficient information.
